(19) United States
(12) Patent Application Publication
(10) Pub. No.: US 2017/0266222 A1
(43) Pub. Date: Sep. 21, 2017

(54) MODULATION OF CHRFAM7A FOR ANTI-INFLAMMATORY THERAPIES

(71) Applicant: THE REGENTS OF THE UNIVERSITY OF CALIFORNIA, Oakland, CA (US)

(72) Inventors: Andrew Baird, San Diego, CA (US); Raul Coimbra, San Diego, CA (US); Todd Costantini, San Diego, CA (US); Brian Eliceiri, Carlsbad, CA (US)

(21) Appl. No.: 15/425,595

(22) Filed: Feb. 6, 2017

Related U.S. Application Data
(60) Provisional application No. 62/291,702, filed on Feb. 5, 2016.

(51) Int. Cl. A61K 31/739; A61K 38/17; C07K 16/44

(52) U.S. Cl. CPC A61K 31/739; C07K 16/44; A61K 38/1787

(57) ABSTRACT

The invention provides a pharmaceutical composition and methods of use thereof, for anti-inflammatory treatment, by altering expression and/or activity of CHRFAM7A, in leukocytes, as well as in epithelial cells.

### CROSS REFERENCES TO RELATED APPLICATIONS

This application claims priority to U.S. Provisional Application No. 62/291,702, filed Feb. 5, 2016, the entire contents of which are incorporated by reference herewith.

### GOVERNMENT SPONSORSHIP

This invention was made with government support under Grants CA170140 and GM078421 awarded by National Institutes of Health, and Grant W81XWH-10-1-0527 awarded by Department of Defense. The government has certain rights in the invention.

### FIELD OF THE INVENTION

Aspects of the invention are generally related to the field of molecular biology, diagnostics, and therapy. More specifically, the invention relates to CHRFAM7A expression which alters the inflammation response.

### BACKGROUND OF THE INVENTION

While there are many genes that humans share with other species, some genes are species-specific and are unique to humans. There are over 300 human-specific genes that have been identified to date and may be associated with complex human disease.

There is a general consensus that the neuroinflammatory response during infection, inflammation, tissue repair and regeneration, is mediated by the α7-acetylcholine receptor (α7nAChR/dupα7). One documented genetic change in the region of the α7nAchR is the emergence of a new CHRFAM7A gene that is distinct but structurally related to α7nAChR/CHRNA7. Formed by a partial duplication of exons 5-10 of the human α7nAChR/CHRNA7 gene, CHRFAM7A is a rearrangement and in-frame fusion of these exons with those of another partially duplicated and rearranged human kinase gene (FAM/ULK4) that originated from chromosome 3. The resulting CHRFAM7 gene has five duplicated exons (exons A-E) of the FAM7 gene rearranged 5′ to the six duplicated exons (exons 5-10) of CHRNA7 to form a new hybrid gene called, CHRFAM7A. There is differential expression of CHRFAM7A in human leukocytes with increased expression of CHRFAM7A compared to CHRNA7. CHRFAM7A expression also alters the expression of the α7nAchR on leukocytes and alters bungarotoxin binding. The ligand for CHRFAM7A is unknown.

### SUMMARY OF THE INVENTION

The invention provides a novel therapeutic target for anti-inflammatory treatment in leukocytes for clinical diseases including sepsis, the systemic inflammatory response to injury, and pancreatitis, and/or for anti-inflammatory treatment in epithelium for clinical diseases including sepsis, trauma injury, burn injury, inflammatory bowel disease, necrotizing enterocolitis, enteritis, and infectious colitis. More specifically, the therapeutic target as identified by the invention is CHRFAM7A.

In certain embodiments, the invention provides that the promoter controlling CHRFAM7A expression is modulated by lipopolysaccharide (LPS) and could represent a therapeutic target aimed at attenuating the inflammatory response. In certain embodiments, the invention provides that CHRFAM7A expression alters the expression of CHRNA7 and alters binding to the α7nAchR, suggesting that CHRFAM7A could be a target to alter the leukocyte inflammatory response either directly, or through its ability to alter α7nAchR expression and/or function.

In certain embodiments, the invention provides that CHRFAM7A expression alters leukocyte adhesion based on RNAseq pathway analysis. This could have implications in the leukocyte response to injury and infection

In other embodiments, the invention identifies CHRFAM7A expression in gut epithelial cells and characterizes its promoter which is modulated by LPS. The invention further provides that CHRFAM7A mediates differential responsiveness to LPS compared to the α7nAchR gene CHRNA7, suggesting that CHRFAM7A could mediate the gut epithelial response to inflammation and represent a novel therapeutic target.

In yet other embodiments, the invention provides that CHRFAM7A increases expression and/or function of α7nAchR. Treatments increasing CHRFAM7A expression would modulate α7nAchR expression and hence alter the inflammation response.

These and other aspects of the present invention will be apparent to those of ordinary skill in the art in the following description, claims and drawings.

### BRIEF DESCRIPTION OF THE DRAWINGS

This patent application file contains at least one drawing executed in color. Copies of this patent application with color drawing(s) will be provided by the Office upon request and payment of the necessary fee.

FIGS. 1A-1C. Expression of CHRNA7 and CHRFAM7A in human leukocytes. RT-PCR of mRNA from human leukocytes cells isolated from 7 patients (lanes 1-7) was used to identify the presence of (FIG. 1A) the duplicate α7-nicotinic acetyl choline receptor (CHRFAM7A), (FIG. 1B) the human α7-nicotinic acetyl choline receptor (CHRNA7), or (FIG. 1C) GAPDH. Arrows show the expected size of the amplified sequences which was confirmed by assessing the size after amplification of the cognate plasmid (P). FIG. 1D shows the results from quantitative RT-PCR for both CHRFAM7A and CHRNA7 which was used to determine the levels of gene expression as measured against a plasmid standard curve of each gene and then expressed as copy number/μg of total mRNA.

FIGS. 2A-2C. Identification of CHRFAM7A in THP12 Cells. FIG. 2A: 5′RACE of human THP1 cells identified 11 of 14 CHRFAM7A transcripts sequenced as initiating at −446 bp from the CHRFAM7A open reading frame (A of the ATG=0) (SEQ ID NO:1). Translation of this sequence established that the deduced CHRFAM7A sequence (FIG. 2B) (SEQ ID NO:2) has a unique 27 amino acid sequence that distinguishes CHRFAM7A from the amino terminus of CHRNA7 (FIG. 2C) (SEQ ID NO:3) and the common 386 amino acid sequence shared by both CHRFAM7A and CHRNA7 (FIG. 2D) (SEQ ID NO:4). The CHRFAM7A exons and the amino acid sequence they encode are shown in blue and red while the CHRNA7 sequence is shown in black triplet codons (FIG. 2A) and amino acids (FIG. 2C).

FIGS. 3A-3H. Regulation of CHRNA7 and CHRFAM7A in Leukocyte Lines. mRNA in (1) HL60, (2) RPMI8226. (3) U937, (4) HEL92, (5) Jurkat, (6) ARH77 or (7) THP1 cells was probed for the presence of CHRFAM7A (FIG. 3A), CHRNA7 (FIG. 3B) and GAPDH (FIG. 3C) by RT-PCR and the amplicon compared to that generated with their respective plasmid standard encoding transcript 1 of human CHRFAM7A, transcript 1 of human CHRNA7 or human GAPDH. The differences in gene expression were quantified in triplicate cultures cells expressed as mean+/−standard deviations after the relative amounts of CHRNA7 and CHRFAM7A gene expression measured by quantitative RT-PCR and normalized to the levels of gene expression in HL60 cells (FIG. 3D). The ratio of CHRFAM7A/CHRNA7 gene expression (FIG. 3E) varies over 10,000 fold between different cell types (e.g. Jurkat vs. THP1) but when cells were treated for 3 hours with 100 ng/ml lipopolysaccharide (LPS) both genes respond equally (FIG. 3F). A schematic representation of 5′UTR-CHRFAM7A (FIG. 3G) shows potential transcription factor binding sites identified by consensus sequence analyses. The f2400, f1800, f1000 and f500 bp fragments of the 5′UTR were used in promoter analyses in control and LPS stimulated THP1 cells (FIG. 3H) to show luciferase expression up-regulated by the f500 fragment in THP1 cells but that inhibitory elements are active in further 5′ extensions. The overall luciferase signal is inhibited in cells treated with LPS.

FIGS. 4A-4E. Biological Consequence of CHRFAM7A Expression. As shown in FIG. 4A, THP1 cells appear as characteristic mononuclear cells growing in suspension but after CHRFAM7A transfection (FIG. 4B) acquire a preponderance for an adhesion phenotype demonstrating that CHRFAM7A is biologically active. As expected the parental THP1 cells specifically bind bungarotoxin because they express CHRNA7 and differential binding can be measured by flow cytometry with labeled bungarotoxin (FIG. 4C). Flow cytometry also shows that transduction of THP1 cells with CHRFAM7A increased bungarotoxin binding (FIG. 4D) when compared to transfection of THP1 cells with GFP vectors. The difference in specific bungarotoxin binding can be quantified by measuring the increase in mean fluorescence after incubating cells with labeled ligand (FIG. 4E). It is likely attributable to induced expression of CHRNA7.

FIGS. 5A-5C. KEGG Pathway Analyses of CHRFAM7A Expression in THP1 cells. The differentially expressed genes in cancer (FIG. 5A), focal adhesion (FIG. 5B) and leukocyte trans-endothelial migration (FIG. 5C) are shown.

FIG. 6. CHRFAM7A Specification of the Vagus-Mediated Inflammatory Response of Human Leukocytes. The identification of a gene encoding a human-specific subunit of α7nAChR raises the possibility that in humans, the canonical vagus nerve regulation of inflammation by activating the the cell surface α7nAChR homopentamer on human leukocytes may be mediated by receptors composed of CHRFAM7A or both CHRFAM7A and CHRNA7A subunits leading to altered ligand tropism, binding kinetics and cell responsiveness.

FIGS. 7A-7E. Identification of CHRFAM7A. FIG. 7A. RT-PCR of CHRFAM7A, CHRNA7 and GAPDH of mRNA isolated from human epithelial cell lines reveals the presence of transcripts in human embryonic kidney (HEK2931 and HEK293W), liver cancer (SKHep), ovarian cancer (OvCar8 and OvCar8-6), pancreatic (PANC1, DU145), colon cancer (HCT116), prostate cancer (PC3) and lung cancer (H1299) epithelial cells. FIG. 7B Overlapping the 3′ and 5′ sequences obtained with the primer (bold) from CaCo2 cells revealed the nucleotide sequence of human epidermal CHRFAM7A (SEQ ID NO:5) that has the exons A (blue) and B (red) of FAM7 and the exons 5-10 of CHRNA7 (Black) which when translated, reveals the unique 27 amino acid sequence of CHRFAM7A (FIG. 7C) (SEQ ID NO:6) that distinguishes the human specific gene from the amino termini found in CHRNA7 (FIG. 7D) (SEQ ID NO:7) The CHRFAM7A PCR primers shown in the text were selected to detect the nucleotide sequences that encode the unique CHRFAM7A peptide (FIG. 7C) whilst the CHRNA7A primers enable detection of transcript variants 1 and 2 mRNAs which encode α7nACHRs that differ by the inclusion of 28 amino acids their amino terminus. The common 386 amino acid sequence shared by both CHRFAM7A and both CHRNA7s is shown in FIG. 7E (SEQ ID NO:8).

FIGS. 8A-8D. CHRFAM7A is Expressed in Epithelial Cells: PC3 cells were transfected with plasmid encoding a CHRFAM7A-DDK fusion protein and the following day lysed and immunoblotted with antibodies to the DDK tag (FIG. 8A). Un-transfected cells were used as control and molecular weights (kDa) determined with molecular weights standards. In FIG. 8 B RT-PCR was used to detect CHRNA7 and CHRFAM7A expression in gut (1) CaCo2, (2) KM12. (3) HT29, (4) KM20, (5) LS174, (6) HCT116, (7) SW24, (8) Colo205 epithelial cells. In three instances (CaCoT, HCT116T, KM20L) the same cell line from two alternative sources were analyzed. As shown in FIG. 8C and FIG. 8D, both CHRNA7 and CHRFAM7A were measured by quantitative RT-PCR and the relative expression levels compared to that measured in CaCo2 cells.

FIGS. 9A-9D. Differential Regulation of CHRNA7 and CHRFAM7A by LPS treatment of Gut Epithelial Cell. Triplicate cultures of (1) CaCo2, (2) KM12. (3) HT29, (4) KM20, (5) LS174, (6) HCT116, (7) SW24, (8) Colo205 and (9) FHs epithelial cells were treated for 3 hours with lipopolysaccharide (LPS) as described in the text. FIG. 9A shows the effects of LPS on CHRNA7 gene expression measured by qPCR, normalized to GAPDH and changes from controls assessed using the ΔΔCt method. The cDNA prepared from the same cell lysates were assessed for CHRFAM7A gene expression (FIG. 9B). In FIG. 9C and FIG. 9D, the ratio of CHRFAM7A expression to CHRNA7 in control and LPS stimulated cells were compared.

FIGS. 10A-10E. Identification of the CHRFAM7A promoter. FIG. 10A. RT-PCR was used to demonstrate that FHs cells express CHRFAM7A and FIG. 10B q-PCR used to show that CHRFAM7A gene expression increases with a 3 hr treatment of cells with 100 ng/ml LPS. FIG. 10C presents a schematic representation of UTR of CHRFAM7A that was used to assess potential transcription factor binding sites in the 2400, 1800, 1000 and 500 bp fragments used in promoter analyses of control (FIG. 10D) and LPS-stimulated (FIG. 10E) cells. Luciferase expression was measured by spectrophotometry and normalized to control cells transduced with the backbone pGL4 vector and no promoter sequence.

### DETAILED DESCRIPTION OF THE INVENTION

The practice of the present invention will employ, unless otherwise indicated, conventional techniques of nanotechnology, nano-engineering, molecular biology (including recombinant techniques), microbiology, cell biology, biochemistry, immunology, and pharmacology, which are within the skill of the art. Such techniques are explained fully in the literature, such as, Molecular Cloning: A Laboratory Manual, 2^{nd} ed. (Sambrook et al., 1989); Oligonucleotide Synthesis (M. J. Gait, ed., 1984); Animal Cell Culture (R. I. Freshney, ed., 1987); Methods in Enzymology (Academic Press, Inc.); Current Protocols in Molecular Biology (F. M. Ausubel et al., eds., 1987, and periodic updates); PCR: The Polymerase Chain Reaction (Mullis et al., eds., 1994); and Remington, The Science and Practice of Pharmacy, 20^{th} ed., (Lippincott, Williams & Wilkins 2003).

It is to be understood that the invention is not limited in its application to the details of and the arrangement of components set forth in the following description. It is also to be understood that this invention is not limited to particular oligonucleotide probes, methods, compositions, reaction mixtures, kits, systems, computers, or computer readable media, which can, of course, vary. It is further to be understood that the terminology used herein is for the purpose of describing particular embodiments only, and is not intended to be limiting. Further, unless defined otherwise, all technical and scientific terms used herein have the same meaning as commonly understood by one of ordinary skill in the art to which this invention pertains. Each of the references cited herein is incorporated by reference in its entirety. In describing and claiming the present invention, the following terminology and grammatical variants will be used in accordance with the definitions set forth below.

### A. Definitions

To facilitate understanding of the invention, a number of terms and abbreviations as used herein are defined below as follows:

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

As used herein, the term “patient” or “subject” refers to an animal, a non-human mammal or a human. As used herein, “animals” include a pet, a farm animal, an economic animal, a sport animal and an experimental animal, such as a cat, a dog, a horse, a cow, an ox, a pig, a donkey, a sheep, a lamb, a goat, a mouse, a rabbit, a chicken, a duck, a goose, a primate, including a monkey and a chimpanzee.

As used herein, the term “agent” or “therapeutic agent” means any naturally occurring or synthesized substance, element, molecule, functional group, compound, fragments thereof or moiety capable of modulating expression or activity of CHRFAM7A, CHRNA7, α7nAchR, or other human-specific genes (HSGs) or taxonomically-restricted genes (TRGs) in leukocytes, including but not limited to, small molecule, biologics, peptides, proteins, or antibodies. Examples of compounds include lipopolysaccharides (LPSs).

The term “antibody” as used herein encompasses monoclonal antibodies (including full length monoclonal antibodies), polyclonal antibodies, multi-specific antibodies (e.g., bi-specific antibodies), and antibody fragments so long as they exhibit the desired biological activity of binding to a target antigenic site and its isoforms of interest. The term “antibody fragments” comprise a portion of a full length antibody, generally the antigen binding or variable region thereof. The term “antibody” as used herein encompasses any antibodies derived from any species and resources, including but not limited to, human antibody, rat antibody, mouse antibody, rabbit antibody, and so on, and can be synthetically made or naturally-occurring.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The “monoclonal antibodies” may also be isolated from phage antibody libraries using the techniques known in the art.

The monoclonal antibodies herein include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. As used herein, a “chimeric protein” or “fusion protein” comprises a first polypeptide operatively linked to a second polypeptide. Chimeric proteins may optionally comprise a third, fourth or fifth or other polypeptide operatively linked to a first or second polypeptide. Chimeric proteins may comprise two or more different polypeptides. Chimeric proteins may comprise multiple copies of the same polypeptide. Chimeric proteins may also comprise one or more mutations in one or more of the polypeptides. Methods for making chimeric proteins are well known in the art.

An “isolated” antibody is one that has been identified and separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-polyacrylamide gel electrophoresis under reducing or non-reducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

In order to avoid potential immunogenicity of the monoclonal antibodies in humans, the monoclonal antibodies that have the desired function are preferably human or humanized. “Humanized” forms of non-human (e.g., murine) antibodies are chimeric antibodies that contain minimal sequence derived from non-human immunoglobulin. For the most part, humanized antibodies are human immunoglobulins (recipient antibody) in which hyper variable region residues of the recipient are replaced by hyper variable region residues from a non-human species (donor antibody) such as mouse, rat, rabbit or nonhuman primate having the desired specificity, affinity, and capacity. Furthermore, humanized antibodies may comprise residues that are not found in the recipient antibody or in the donor antibody. These modifications are made to further refine antibody performance.

The therapeutic agent may also refer to any oligonucleotides (antisense oligonucleotide agents), polynucleotides (e.g. therapeutic DNA), ribozymes, DNA aptamers, dsRNAs, siRNA, RNAi, and/or gene therapy vectors. The term “antisense oligonucleotide agent” refers to short synthetic segments of DNA or RNA, usually referred to as oligonucleotides, which are designed to be complementary to a sequence of a specific mRNA to inhibit the translation of the targeted mRNA by binding to a unique sequence segment on the mRNA. Antisense oligonucleotides are often developed and used in the antisense technology. The term “antisense technology” refers to a drug-discovery and development technique that involves design and use of synthetic oligonucleotides complementary to a target mRNA to inhibit production of specific disease-causing proteins. Antisense technology permits design of drugs, called antisense oligonucleotides, which intervene at the genetic level and inhibit the production of disease-associated proteins. Antisense oligonucleotide agents are developed based on genetic information.

As an alternative to antisense oligonucleotide agents, ribozymes or double stranded RNA (dsRNA), RNA interference (RNAi), and/or small interfering RNA (siRNA), can also be used as therapeutic agents for regulation of gene expression in cells. As used herein, the term “ribozyme” refers to a catalytic RNA-based enzyme with ribonuclease activity that is capable of cleaving a single-stranded nucleic acid, such as an mRNA, to which it has a complementary region. Ribozymes can be used to catalytically cleave target mRNA transcripts to thereby inhibit translation of target mRNA. The term “dsRNA,” as used herein, refers to RNA hybrids comprising two strands of RNA. The dsRNAs can be linear or circular in structure. The dsRNA may comprise ribonucleotides, ribonucleotide analogs, such as 2′-O-methyl ribosyl residues, or combinations thereof. The term “RNAi” refers to RNA interference or post-transcriptional gene silencing (PTGS). The term “siRNA” refers to small dsRNA molecules (e.g., 21-23 nucleotides) that are the mediators of the RNAi effects. RNAi is induced by the introduction of long dsRNA (up to 1-2 kb) produced by in vitro transcription, and has been successfully used to reduce gene expression in variety of organisms. In mammalian cells, RNAi uses siRNA (e.g. 22 nucleotides long) to bind to the RNA-induced silencing complex (RISC), which then binds to any matching mRNA sequence to degrade target mRNA, thus, silences the gene.

“Amplification” refers to any known procedure for obtaining multiple copies of a target nucleic acid or its complement, or fragments thereof. The multiple copies may be referred to as amplicons or amplification products. Amplification, in the context of fragments, refers to production of an amplified nucleic acid that contains less than the complete target nucleic acid or its complement, e.g., produced by using an amplification oligonucleotide that hybridizes to, and initiates polymerization from, an internal position of the target nucleic acid. Known amplification methods include, for example, replicase-mediated amplification, polymerase chain reaction (PCR), reverse transcription polymerase chain reaction (RT-PCR), ligase chain reaction (LCR), strand-displacement amplification (SDA), and transcription-mediated or transcription-associated amplification. Amplification is not limited to the strict duplication of the starting molecule. For example, the generation of multiple cDNA molecules from RNA in a sample using reverse transcription (RT)-PCR is a form of amplification. Furthermore, the generation of multiple RNA molecules from a single DNA molecule during the process of transcription is also a form of amplification. During amplification, the amplified products can be labeled using, for example, labeled primers or by incorporating labeled nucleotides.

“Amplicon” or “amplification product” refers to the nucleic acid molecule generated during an amplification procedure that is complementary or homologous to a target nucleic acid or a region thereof. Amplicons can be double stranded or single stranded and can include DNA, RNA or both. Methods for generating amplicons are known to those skilled in the art.

“Codon” refers to a sequence of three nucleotides that together form a unit of genetic code in a nucleic acid.

“Codon of interest” refers to a specific codon in a target nucleic acid that has diagnostic or therapeutic significance (e.g. an allele associated with viral genotype/subtype or drug resistance).

“Complementary” or “complement thereof” means that a contiguous nucleic acid base sequence is capable of hybridizing to another base sequence by standard base pairing (hydrogen bonding) between a series of complementary bases. Complementary sequences may be completely complementary (i.e. no mismatches in the nucleic acid duplex) at each position in an oligomer sequence relative to its target sequence by using standard base pairing (e.g., G:C, A:T or A:U pairing) or sequences may contain one or more positions that are not complementary by base pairing (e.g., there exists at least one mismatch or unmatched base in the nucleic acid duplex), but such sequences are sufficiently complementary because the entire oligomer sequence is capable of specifically hybridizing with its target sequence in appropriate hybridization conditions (i.e. partially complementary). Contiguous bases in an oligomer are typically at least 80%, preferably at least 90%, and more preferably completely complementary to the intended target sequence.

“Configured to” or “designed to” denotes an actual arrangement of a nucleic acid sequence configuration of a referenced oligonucleotide. For example, a primer that is configured to generate a specified amplicon from a target nucleic acid has a nucleic acid sequence that hybridizes to the target nucleic acid or a region thereof and can be used in an amplification reaction to generate the amplicon. Also as an example, an oligonucleotide that is configured to specifically hybridize to a target nucleic acid or a region thereof has a nucleic acid sequence that specifically hybridizes to the referenced sequence under stringent hybridization conditions.

“Downstream” means further along a nucleic acid sequence in the direction of sequence transcription or read out.

“Upstream” means further along a nucleic acid sequence in the direction opposite to the direction of sequence transcription or read out.

“Polymerase chain reaction” (PCR) generally refers to a process that uses multiple cycles of nucleic acid denaturation, annealing of primer pairs to opposite strands (forward and reverse), and primer extension to exponentially increase copy numbers of a target nucleic acid sequence. In a variation called RT-PCR, reverse transcriptase (RT) is used to make a complementary DNA (cDNA) from mRNA, and the cDNA is then amplified by PCR to produce multiple copies of DNA. There are many permutations of PCR known to those of ordinary skill in the art.

“Position” refers to a particular amino acid or amino acids in a nucleic acid sequence.

“Primer” refers to an enzymatically extendable oligonucleotide, generally with a defined sequence that is designed to hybridize in an antiparallel manner with a complementary, primer-specific portion of a target nucleic acid. A primer can initiate the polymerization of nucleotides in a template-dependent manner to yield a nucleic acid that is complementary to the target nucleic acid when placed under suitable nucleic acid synthesis conditions (e.g. a primer annealed to a target can be extended in the presence of nucleotides and a DNA/RNA polymerase at a suitable temperature and pH). Suitable reaction conditions and reagents are known to those of ordinary skill in the art. A primer is typically single stranded for maximum efficiency in amplification, but may alternatively be double stranded. If double stranded, the primer is generally first treated to separate its strands before being used to prepare extension products. The primer generally is sufficiently long to prime the synthesis of extension products in the presence of the inducing agent (e.g. polymerase). Specific length and sequence will be dependent on the complexity of the required DNA or RNA targets, as well as on the conditions of primer use such as temperature and ionic strength. Preferably, the primer is about 5-100 nucleotides. Thus, a primer can be, e.g., 5, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 65, 70, 75, 80, 85, 90, 95 or 100 nucleotides in length. A primer does not need to have 100% complementarity with its template for primer elongation to occur; primers with less than 100% complementarity can be sufficient for hybridization and polymerase elongation to occur. A primer can be labeled if desired. The label used on a primer can be any suitable label, and can be detected by, for example, spectroscopic, photochemical, biochemical, immunochemical, chemical, or other detection means. A labeled primer therefore refers to an oligomer that hybridizes specifically to a target sequence in a nucleic acid, or in an amplified nucleic acid, under conditions that promote hybridization to allow selective detection of the target sequence.

A primer nucleic acid can be labeled, if desired, by incorporating a label detectable by, e.g., spectroscopic, photochemical, biochemical, immunochemical, chemical, or other techniques. To illustrate, useful labels include radioisotopes, fluorescent dyes, electron-dense reagents, enzymes (as commonly used in ELISAs), biotin, or haptens and proteins for which antisera or monoclonal antibodies are available. Many of these and other labels are described further herein and/or are otherwise known in the art. One of skill in the art will recognize that, in certain embodiments, primer nucleic acids can also be used as probe nucleic acids.

“Region” refers to a portion of a nucleic acid wherein said portion is smaller than the entire nucleic acid.

“Region of interest” refers to a specific sequence of a target nucleic acid that includes all codon positions having at least one single nucleotide substitution mutation associated with a genotype and/or subtype that are to be amplified and detected, and all marker positions that are to be amplified and detected, if any.

“RNA-dependent DNA polymerase” or “reverse transcriptase” (“RT”) refers to an enzyme that synthesizes a complementary DNA copy from an RNA template. All known reverse transcriptases also have the ability to make a complementary DNA copy from a DNA template; thus, they are both RNA- and DNA-dependent DNA polymerases. RTs may also have an RNAse H activity. A primer is required to initiate synthesis with both RNA and DNA templates.

“DNA-dependent DNA polymerase” is an enzyme that synthesizes a complementary DNA copy from a DNA template. Examples are DNA polymerase I from E. coli, bacteriophage T7 DNA polymerase, or DNA polymerases from bacteriophages T4, Phi-29, M2, or T5. DNA-dependent DNA polymerases may be the naturally occurring enzymes isolated from bacteria or bacteriophages or expressed recombinantly, or may be modified or “evolved” forms which have been engineered to possess certain desirable characteristics, e.g., thermostability, or the ability to recognize or synthesize a DNA strand from various modified templates. All known DNA-dependent DNA polymerases require a complementary primer to initiate synthesis. It is known that under suitable conditions a DNA-dependent DNA polymerase may synthesize a complementary DNA copy from an RNA template. RNA-dependent DNA polymerases typically also have DNA-dependent DNA polymerase activity.

“DNA-dependent RNA polymerase” or “transcriptase” is an enzyme that synthesizes multiple RNA copies from a double-stranded or partially double-stranded DNA molecule having a promoter sequence that is usually double-stranded. The RNA molecules (“transcripts”) are synthesized in the 5′-to-3′ direction beginning at a specific position just downstream of the promoter. Examples of transcriptases are the DNA-dependent RNA polymerase from E. coli and bacteriophages T7, T3, and SP6.

A “sequence” of a nucleic acid refers to the order and identity of nucleotides in the nucleic acid. A sequence is typically read in the 5′ to 3′ direction. The terms “identical” or percent “identity” in the context of two or more nucleic acid or polypeptide sequences, refer to two or more sequences or subsequences that are the same or have a specified percentage of amino acid residues or nucleotides that are the same, when compared and aligned for maximum correspondence, e.g., as measured using one of the sequence comparison algorithms available to persons of skill or by visual inspection. Exemplary algorithms that are suitable for determining percent sequence identity and sequence similarity are the BLAST programs, which are described in, e.g., Altschul et al. (1990) “Basic local alignment search tool” J. Mol. Biol. 215:403-410, Gish et al. (1993) “Identification of protein coding regions by database similarity search” Nature Genet. 3:266-272, Madden et al. (1996) “Applications of network BLAST server” Meth. Enzymol. 266:131-141, Altschul et al. (1997) “Gapped BLAST and PSI-BLAST: a new generation of protein database search programs” Nucleic Acids Res. 25:3389-3402, and Zhang et al. (1997) “PowerBLAST: A new network BLAST application for interactive or automated sequence analysis and annotation” Genome Res. 7:649-656, which are each incorporated by reference. Many other optimal alignment algorithms are also known in the art and are optionally utilized to determine percent sequence identity.

A “label” refers to a moiety attached (covalently or non-covalently), or capable of being attached, to a molecule, which moiety provides or is capable of providing information about the molecule (e.g., descriptive, identifying, etc. information about the molecule) or another molecule with which the labeled molecule interacts (e.g., hybridizes, etc.). Exemplary labels include fluorescent labels (including, e.g., quenchers or absorbers), weakly fluorescent labels, non-fluorescent labels, colorimetric labels, chemiluminescent labels, bioluminescent labels, radioactive labels, mass-modifying groups, antibodies, antigens, biotin, haptens, enzymes (including, e.g., peroxidase, phosphatase, etc.), and the like.

A “linker” refers to a chemical moiety that covalently or non-covalently attaches a compound or substituent group to another moiety, e.g., a nucleic acid, an oligonucleotide probe, a primer nucleic acid, an amplicon, a solid support, or the like. For example, linkers are optionally used to attach oligonucleotide probes to a solid support (e.g., in a linear or other logic probe array). To further illustrate, a linker optionally attaches a label (e.g., a fluorescent dye, a radioisotope, etc.) to an oligonucleotide probe, a primer nucleic acid, or the like. Linkers are typically at least bifunctional chemical moieties and in certain embodiments, they comprise cleavable attachments, which can be cleaved by, e.g., heat, an enzyme, a chemical agent, electromagnetic radiation, etc. to release materials or compounds from, e.g., a solid support. A careful choice of linker allows cleavage to be performed under appropriate conditions compatible with the stability of the compound and assay method. Generally a linker has no specific biological activity other than to, e.g., join chemical species together or to preserve some minimum distance or other spatial relationship between such species. However, the constituents of a linker may be selected to influence some property of the linked chemical species such as three-dimensional conformation, net charge, hydrophobicity, etc. Exemplary linkers include, e.g., oligopeptides, oligonucleotides, oligopolyamides, oligoethyleneglycerols, oligoacrylamides, alkyl chains, or the like. Additional description of linker molecules is provided in, e.g., Hermanson, Bioconjugate Techniques, Elsevier Science (1996), Lyttle et al. (1996) Nucleic Acids Res. 24(14):2793, Shchepino et al. (2001) Nucleosides, Nucleotides, & Nucleic Acids 20:369, Doronina et al (2001) Nucleosides, Nucleotides, & Nucleic Acids 20:1007, Trawick et al. (2001) Bioconjugate Chem. 12:900, Olejnik et al. (1998) Methods in Enzymology 291:135, and Pljevaljcic et al. (2003) J. Am. Chem. Soc. 125(12):3486, all of which are incorporated by reference.

“Fragment” refers to a piece of contiguous nucleic acid that contains fewer nucleotides than the complete nucleic acid.

“Hybridization,” “annealing,” “selectively bind,” or “selective binding” refers to the base-pairing interaction of one nucleic acid with another nucleic acid (typically an antiparallel nucleic acid) that results in formation of a duplex or other higher-ordered structure (i.e. a hybridization complex). The primary interaction between the antiparallel nucleic acid molecules is typically base specific, e.g., A/T and G/C. It is not a requirement that two nucleic acids have 100% complementarity over their full length to achieve hybridization. Nucleic acids hybridize due to a variety of well characterized physio-chemical forces, such as hydrogen bonding, solvent exclusion, base stacking and the like. An extensive guide to the hybridization of nucleic acids is found in Tijssen (1993) Laboratory Techniques in Biochemistry and Molecular Biology—Hybridization with Nucleic Acid Probes part I chapter 2, “Overview of principles of hybridization and the strategy of nucleic acid probe assays,” (Elsevier, New York), as well as in Ausubel (Ed.) Current Protocols in Molecular Biology, Volumes I, II, and III, 1997, which is incorporated by reference.

The term “attached” or “conjugated” refers to interactions and/or states in which material or compounds are connected or otherwise joined with one another. These interactions and/or states are typically produced by, e.g., covalent bonding, ionic bonding, chemisorption, physisorption, and combinations thereof.

A “composition” refers to a combination of two or more different components. In certain embodiments, for example, a composition includes one or more oligonucleotide probes in solution.

The term “derivative” refers to a chemical substance related structurally to another substance, or a chemical substance that can be made from another substance (i.e., the substance it is derived from), e.g., through chemical or enzymatic modification. To illustrate, oligonucleotide probes are optionally conjugated with biotin or a biotin derivative. To further illustrate, one nucleic acid can be “derived” from another through processes, such as chemical synthesis based on knowledge of the sequence of the other nucleic acid, amplification of the other nucleic acid, or the like.

“Nucleic acid” or “nucleic acid molecule” refers to a multimeric compound comprising two or more covalently bonded nucleosides or nucleoside analogs having nitrogenous heterocyclic bases, or base analogs, where the nucleosides are linked together by phosphodiester bonds or other linkages to form a polynucleotide. Nucleic acids include RNA, DNA, or chimeric DNA-RNA polymers or oligonucleotides, and analogs thereof. A nucleic acid backbone can be made up of a variety of linkages, including one or more of sugar-phosphodiester linkages, peptide-nucleic acid bonds, phosphorothioate linkages, methylphosphonate linkages, or combinations thereof. Sugar moieties of the nucleic acid can be ribose, deoxyribose, or similar compounds having known substitutions (e.g. 2′-methoxy substitutions and 2′-halide substitutions). Nitrogenous bases can be conventional bases (A, G, C, T, U) or analogs thereof (e.g., inosine, 5-methylisocytosine, isoguanine). A nucleic acid can comprise only conventional sugars, bases, and linkages as found in RNA and DNA, or can include conventional components and substitutions (e.g., conventional bases linked by a 2′-methoxy backbone, or a nucleic acid including a mixture of conventional bases and one or more base analogs). Nucleic acids can include “locked nucleic acids” (LNA), in which one or more nucleotide monomers have a bicyclic furanose unit locked in an RNA mimicking sugar conformation, which enhances hybridization affinity toward complementary sequences in single-stranded RNA (ssRNA), single-stranded DNA (ssDNA), or double-stranded DNA (dsDNA). Nucleic acids can include modified bases to alter the function or behavior of the nucleic acid (e.g., addition of a 3′-terminal dideoxynucleotide to block additional nucleotides from being added to the nucleic acid). Synthetic methods for making nucleic acids in vitro are well known in the art although nucleic acids can be purified from natural sources using routine techniques. Nucleic acids can be single-stranded or double-stranded.

A nucleic acid is typically single-stranded or double-stranded and will generally contain phosphodiester bonds, although in some cases, as outlined, herein, nucleic acid analogs are included that may have alternate backbones, including, for example and without limitation, phosphoramide (Beaucage et al. (1993) Tetrahedron 49(10):1925 and references therein; Letsinger (1970) J. Org. Chem. 35:3800; Sprinzl et al. (1977) Eur. J. Biochem. 81:579; Letsinger et al. (1986) Nucl. Acids Res. 14: 3487; Sawai et al. (1984) Chem. Lett. 805; Letsinger et al. (1988) J. Am. Chem. Soc. 110:4470; and Pauwels et al. (1986) Chemica Scripta 26: 1419, which are each incorporated by reference), phosphorothioate (Mag et al. (1991) Nucleic Acids Res. 19:1437; and U.S. Pat. No. 5,644,048, which are both incorporated by reference), phosphorodithioate (Briu et al. (1989) J. Am. Chem. Soc. 111:2321, which is incorporated by reference), O-methylphosphoroamidite linkages (see Eckstein, Oligonucleotides and Analogues: A Practical Approach, Oxford University Press (1992), which is incorporated by reference), and peptide nucleic acid backbones and linkages (see, Egholm (1992) J. Am. Chem. Soc. 114:1895; Meier et al. (1992) Chem. Int. Ed. Engl. 31:1008; Nielsen (1993) Nature 365:566; and Carlsson et al. (1996) Nature 380:207, which are each incorporated by reference). Other analog nucleic acids include those with positively charged backbones (Denpcy et al. (1995) Proc. Natl. Acad. Sci. USA 92:6097, which is incorporated by reference); non-ionic backbones (U.S. Pat. Nos. 5,386,023, 5,637,684, 5,602,240, 5,216,141 and 4,469,863; Angew (1991) Chem. Intl. Ed. English 30: 423; Letsinger et al. (1988) J. Am. Chem. Soc. 110:4470; Letsinger et al. (1994) Nucleoside & Nucleotide 13:1597; Chapters 2 and 3, ASC Symposium Series 580, “Carbohydrate Modifications in Antisense Research”, Ed. Y. S. Sanghvi and P. Dan Cook; Mesmaeker et al. (1994) Bioorganic & Medicinal Chem: Lett. 4: 395; Jeffs et al. (1994) J. Biomolecular NMR 34:17; and Tetrahedron Lett. 37:743 (1996), which are each incorporated by reference) and non-ribose backbones, including those described in U.S. Pat. Nos. 5,235,033 and 5,034,506, and Chapters 6 and 7, ASC Symposium Series 580, Carbohydrate Modifications in Antisense Research, Ed. Y. S. Sanghvi and P. Dan Cook, which references are each incorporated by reference. Nucleic acids containing one or more carbocyclic sugars are also included within the definition of nucleic acids (see Jenkins et al. (1995) Chem. Soc. Rev. pp 169-176, which is incorporated by reference). Several nucleic acid analogs are also described in, e.g., Rawls, C & E News Jun. 2, 1997 page 35, which is incorporated by reference. These modifications of the ribose-phosphate backbone may be done to facilitate the addition of additional moieties such as labels, or to alter the stability and half-life of such molecules in physiological environments.

In addition to these naturally occurring heterocyclic bases that are typically found in nucleic acids (e.g., adenine, guanine, thymine, cytosine, and uracil), nucleic acid analogs also include those having non-naturally occurring heterocyclic or modified bases, many of which are described, or otherwise referred to, herein. In particular, many non-naturally occurring bases are described further in, e.g., Seela et al. (1991) Helv. Chim. Acta 74:1790, Grein et al. (1994) Bioorg. Med. Chem. Lett. 4:971-976, and Seela et al. (1999) Helv. Chim. Acta 82:1640, which are each incorporated by reference. To further illustrate, certain bases used in nucleotides that act as melting temperature (TO modifiers are optionally included. For example, some of these include 7-deazapurines (e.g., 7-deazaguanine, 7-deazaadenine, etc.), pyrazolo[3,4-d]pyrimidines, propynyl-dN (e.g., propynyl-dU, propynyl-dC, etc.), and the like. See, e.g., U.S. Pat. No. 5,990,303, entitled “SYNTHESIS OF 7-DEAZA-2′-DEOXYGUANOSINE NUCLEOTIDES,” which issued Nov. 23, 1999 to Seela, which is incorporated by reference. Other representative heterocyclic bases include, e.g., hypoxanthine, inosine, xanthine; 8-aza derivatives of 2-aminopurine, 2,6-diaminopurine, 2-amino-6-chloropurine, hypoxanthine, inosine and xanthine; 7-deaza-8-aza derivatives of adenine, guanine, 2-aminopurine, 2,6-diaminopurine, 2-amino-6-chloropurine, hypoxanthine, inosine and xanthine; 6-azacytosine; 5-fluorocytosine; 5-chlorocytosine; 5-iodocytosine; 5-bromocytosine; 5-methylcytosine; 5-propynylcytosine; 5-bromovinyluracil; 5-fluorouracil; 5-chlorouracil; 5-iodouracil; 5-bromouracil; 5-trifluoromethyluracil; 5-methoxymethyluracil; 5-ethynyluracil; 5-propynyluracil, and the like.

Examples of modified bases and nucleotides are also described in, e.g., U.S. Pat. No. 5,484,908, entitled “OLIGONUCLEOTIDES CONTAINING 5-PROPYNYL PYRIMIDINES,” issued Jan. 16, 1996 to Froehler et al., U.S. Pat. No. 5,645,985, entitled “ENHANCED TRIPLE-HELIX AND DOUBLE-HELIX FORMATION WITH OLIGOMERS CONTAINING MODIFIED PYRIMIDINES,” issued Jul. 8, 1997 to Froehler et al., U.S. Pat. No. 5,830,653, entitled “METHODS OF USING OLIGOMERS CONTAINING MODIFIED PYRIMIDINES,” issued Nov. 3, 1998 to Froehler et al., U.S. Pat. No. 6,639,059, entitled “SYNTHESIS OF [2.2.1]BICYCLO NUCLEOSIDES,” issued Oct. 28, 2003 to Kochkine et al., U.S. Pat. No. 6,303,315, entitled “ONE STEP SAMPLE PREPARATION AND DETECTION OF NUCLEIC ACIDS IN COMPLEX BIOLOGICAL SAMPLES,” issued Oct. 16, 2001 to Skouv, and U.S. Pat. Application Pub. No. 2003/0092905, entitled “SYNTHESIS OF [2.2.1]BICYCLO NUCLEOSIDES,” by Kochkine et al. that published May 15, 2003, which are each incorporated by reference.

An “oligonucleotide” or “oligomer” refers to a nucleic acid that includes at least two nucleic acid monomer units (e.g., nucleotides), typically more than three monomer units, and more typically greater than ten monomer units. The exact size of an oligonucleotide generally depends on various factors, including the ultimate function or use of the oligonucleotide. Oligonucleotides are optionally prepared by any suitable method, including, but not limited to, isolation of an existing or natural sequence, DNA replication or amplification, reverse transcription, cloning and restriction digestion of appropriate sequences, or direct chemical synthesis by a method such as the phosphotriester method of Narang et al. (1979) Meth. Enzymol. 68:90-99; the phosphodiester method of Brown et al. (1979) Meth. Enzymol. 68:109-151; the diethylphosphoramidite method of Beaucage et al. (1981) Tetrahedron Lett. 22:1859-1862; the triester method of Matteucci et al. (1981) J. Am. Chem. Soc. 103:3185-3191; automated synthesis methods; or the solid support method of U.S. Pat. No. 4,458,066, or other methods known in the art. All of these references are incorporated by reference.

A “mixture” refers to a combination of two or more different components. A “reaction mixture” refers a mixture that comprises molecules that can participate in and/or facilitate a given reaction. An “amplification reaction mixture” refers to a solution containing reagents necessary to carry out an amplification reaction, and typically contains primers, a thermostable DNA polymerase, dNTP's, and a divalent metal cation in a suitable buffer. A reaction mixture is referred to as complete if it contains all reagents necessary to carry out the reaction, and incomplete if it contains only a subset of the necessary reagents. It will be understood by one of skill in the art that reaction components are routinely stored as separate solutions, each containing a subset of the total components, for reasons of convenience, storage stability, or to allow for application-dependent adjustment of the component concentrations, and, that reaction components are combined prior to the reaction to create a complete reaction mixture. Furthermore, it will be understood by one of skill in the art that reaction components are packaged separately for commercialization and that useful commercial kits may contain any subset of the reaction components, which includes the modified primers of the invention.

The term “pharmaceutically active” as used herein refers to the beneficial biological activity of a substance on living matter and, in particular, on cells and tissues of the human body. A “pharmaceutically active agent” or “drug” is a substance that is pharmaceutically active and a “pharmaceutically active ingredient” (API) is the pharmaceutically active substance in a drug.

The term “pharmaceutically acceptable” as used herein means approved by a regulatory agency of the Federal or a state government or listed in the U.S. Pharmacopoeia, other generally recognized pharmacopoeia in addition to other formulations that are safe for use in animals, and more particularly in humans and/or non-human mammals.

The term “pharmaceutically acceptable salt” as used herein refers to acid addition salts or base addition salts of the compounds, such as the multi-drug conjugates, in the present disclosure. A pharmaceutically acceptable salt is any salt which retains the activity of the parent agent or compound and does not impart any deleterious or undesirable effect on a subject to whom it is administered and in the context in which it is administered. Pharmaceutically acceptable salts may be derived from amino acids including, but not limited to, cysteine. Methods for producing compounds as salts are known to those of skill in the art (see, for example, Stahl et al., Handbook of Pharmaceutical Salts: Properties, Selection, and Use, Wiley-VCH; Verlag Helvetica Chimica Acta, Zurich, 2002; Berge et al., J Pharm. Sci. 66: 1, 1977). In some embodiments, a “pharmaceutically acceptable salt” is intended to mean a salt of a free acid or base of an agent or compound represented herein that is non-toxic, biologically tolerable, or otherwise biologically suitable for administration to the subject. See, generally, Berge, et al., J. Pharm. Sci., 1977, 66, 1-19. Preferred pharmaceutically acceptable salts are those that are pharmacologically effective and suitable for contact with the tissues of subjects without undue toxicity, irritation, or allergic response. An agent or compound described herein may possess a sufficiently acidic group, a sufficiently basic group, both types of functional groups, or more than one of each type, and accordingly react with a number of inorganic or organic bases, and inorganic and organic acids, to form a pharmaceutically acceptable salt.

Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, [gamma]-hydroxybutyrates, glycolates, tartrates, and mandelates.

The term “pharmaceutically acceptable carrier” as used herein refers to an excipient, diluent, preservative, solubilizer, emulsifier, adjuvant, and/or vehicle with which an agent or compound, such as a multi-drug conjugate, is administered. Such carriers may be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be a carrier. Methods for producing compositions in combination with carriers are known to those of skill in the art. In some embodiments, the language “pharmaceutically acceptable carrier” is intended to include any and all solvents, dispersion media, coatings, isotonic and absorption delaying agents, and the like, compatible with pharmaceutical administration. The use of such media and agents for pharmaceutically active substances is well known in the art. See, e.g., Remington, The Science and Practice of Pharmacy. 20'″ ed., (Lippincott, Williams & Wilkins 2003). Except insofar as any conventional media or agent is incompatible with the active compound, such use in the compositions is contemplated. Therapeutically Effective Amount: As used herein, the term “therapeutically effective amount” refers to those amounts that, when administered to a particular subject in view of the nature and severity of that subject's disease or condition, will have a desired therapeutic effect, e.g., an amount which will cure, prevent, inhibit, or at least partially arrest or partially prevent a target disease or condition. More specific embodiments are included in the Pharmaceutical Preparations and Methods of Administration section below. In some embodiments, the term “therapeutically effective amount” or “effective amount” refers to an amount of a therapeutic agent that when administered alone or in combination with an additional therapeutic agent to a cell, tissue, or subject is effective to prevent or ameliorate the disease or condition such as a hemolytic disease or condition, or the progression of the disease or condition. A therapeutically effective dose further refers to that amount of the therapeutic agent sufficient to result in amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously.

“Treating” or “treatment” or “alleviation” refers to therapeutic treatment wherein the object is to slow down (lessen) if not cure the targeted pathologic condition or disorder or prevent recurrence of the condition. A subject is successfully “treated” if, after receiving a therapeutic amount of a therapeutic agent, the subject shows observable and/or measurable reduction in or absence of one or more signs and symptoms of the particular disease. Reduction of the signs or symptoms of a disease may also be felt by the patient. A patient is also considered treated if the patient experiences stable disease. In some embodiments, treatment with a therapeutic agent is effective to result in the patients being disease-free 3 months after treatment, preferably 6 months, more preferably one year, even more preferably 2 or more years post treatment. These parameters for assessing successful treatment and improvement in the disease are readily measurable by routine procedures familiar to a physician of appropriate skill in the art.

As used herein, “preventative” treatment is meant to indicate a postponement of development of a disease, a symptom of a disease, or medical condition, suppressing symptoms that may appear, or reducing the risk of developing or recurrence of a disease or symptom. “Curative” treatment includes reducing the severity of or suppressing the worsening of an existing disease, symptom, or condition.

The term “combination” refers to either a fixed combination in one dosage unit form, or a kit of parts for the combined administration where an agent or compound and a combination partner (e.g., another drug as explained below, also referred to as “therapeutic agent” or “co-agent”) may be administered independently at the same time or separately within time intervals, especially where these time intervals allow that the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g., an agent or compound and a combination partner, are both administered to a patient simultaneously in the form of a single entity or dosage. The term “non-fixed combination” means that the active ingredients, e.g., a agent or compound and a combination partner, are both administered to a patient as separate entities either simultaneously, concurrently or sequentially with no specific time limits, wherein such administration provides therapeutically effective levels of the two moieties or compounds in the body of the patient. The latter also applies to cocktail therapy, e.g., the administration of three or more active ingredients.

It is understood that aspects and embodiments of the invention described herein include “consisting” and/or “consisting essentially of” aspects and embodiments.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Other objects, advantages and features of the present invention will become apparent from the following specification taken in conjunction with the accompanying drawings.

The invention provides a novel therapeutic target for anti-inflammatory treatment in leukocytes, as well as in epithelial cells, for clinical diseases including, but not limited to, sepsis, the systemic inflammatory response to injury, pancreatitis, trauma injury, burn injury, inflammatory bowel disease, necrotizing enterocolitis, enteritis, and infectious colitis. In certain embodiments, the invention provides anti-inflammatory treatment by modulating leukocyte and/or epithelial CHRFAM7A expression or activity. In certain embodiments, the invention provides that the promoter controlling CHRFAM7A expression is modulated by a lipopolysaccharide, and other therapeutic agents, for attenuating an inflammatory response.

In certain embodiments, the invention provides that CHRFAM7A expression alters the expression of CHRNA7 and alters binding to the α7nAchR, such that CHRFAM7A alters the leukocyte inflammatory response either directly, or through its ability to alter α7nAchR expression. In certain embodiments, the invention provides that CHRFAM7A expression alters leukocyte adhesion, which is a useful treatment in the leukocyte response to injury and infection.

In some embodiments, the present methods can be used for altering the expression of CHRFAM7A, CHRNA7, α7nAchR, or other human-specific genes (HSGs) or taxonomically-restricted genes (TRGs) in leukocytes of patients. In other embodiments, the present methods can be used for altering the activity of CHRFAM7A, CHRNA7, α7nAchR, or other HSGs or TRGs in leukocytes of patients. The present methods can be used to alter expression or activity of CHRFAM7A, CHRNA7, α7nAchR, or other HSGs or TRGs in leukocytes of patients to any suitable degree. For example, present methods can be used to alter expression or activity of CHRFAM7A, CHRNA7, α7nAchR, or other HSGs or TRGs in leukocytes in a patient by at least 1%, 2%, 5%, 10%, 15%, 20%, 30%, 40%, 50%, 60%, 70%, 80%, 90%, 91%, 92%, 93%, 94%, 95%, 96%, 97%, 98%, 99%, 200%, 500%, 1000%, or more compared to a comparable untreated patient or to the same patient at an untreated stage.

In certain embodiments, the invention identifies CHRFAM7A expression in gut epithelial cells which mediates differential responsiveness to LPS compared to the α7nAchR gene CHRNA7, suggesting that CHRFAM7A could mediate the gut epithelial response to inflammation and represent a novel therapeutic target. In further embodiments, the invention provides that CHRFAM7A increases expression and/or function of α7nAchR. Treatments increasing CHRFAM7A expression modulate α7nAchR expression or activity and hence alter the inflammation response.

In certain embodiments, the present methods provide a pharmaceutical composition and method for treating an inflammatory response in leukocytes for a clinical disease by administering to a subject a pharmaceutical composition which includes an agent that increases the expression and/or activity of CHRFAM7A. In some embodiments the agent alters α7nAchR binding, and in other embodiments it alters leukocyte adhesion.

In yet other embodiments, the present methods provide a pharmaceutical composition and method for treating inflammation in epithelium for a clinical disease by administering to a subject a pharmaceutical composition which includes an agent that increases the expression and/or activity of CHRFAM7A. In some embodiments the agent alters α7nAchR binding.

In certain embodiments the administered agent comprises a lipopolysaccharide (LPS) or a functional fragment thereof. Generally, lipopolysaccharides (LPSs) are composed of three distinct subunits; a core oligosaccharide, which is subdivided into an inner and an outer core; a phospholipid-lipid A; and an outer polysaccharide-O antigen. These LPS subunits can vary. The inner oligosaccharide core typically consists of Kdo (3-deoxy-D-manno-octulosonic acid) and heptose sugars, whereas the outer core displays variations in sugar composition, sugar arrangement and linkage to O antigen. O antigen, in addition to varying in composition, can also have different lengths, ranging from a complete absence of O antigen to more than 100 repeating units of sugar backbones with branching chains.

In certain embodiments, the administered LPS may be modified, with one or more of the subunits being modified. Examples of modifications include, but are not limited to; adding various constituents, such as additional sugars, phosphate groups, phosphoethanolamine groups, or phosphorylcholine groups, to the core oligosaccharide; modifying the O antigen by glycosylation, acetylation, adding phosphoryl constituents, or ligating acidic repeats such as colanic and sialic acids; and changing the phosphorylation pattern or the number of acyl chains esterified to the disaccharide backbone of lipid A.

The agent that modulates the expression or activity of CHRFAM7A, CHRNA7, α7nAchR, or other human-specific genes (HSGs) or taxonomically-restricted genes (TRGs) in leukocytes, may be administered alone or in combination with other active ingredient(s), described herein, and preferably in the form of a pharmaceutical composition, may be administered by a suitable route of delivery, such as oral, parenteral, rectal, nasal, topical, or ocular routes, or by inhalation. In some embodiments, the compositions are formulated for intravenous or oral administration.

For oral administration, the agent, alone or in combination with another active ingredient, may be provided in a solid form, such as a tablet or capsule, or as a solution, emulsion, or suspension. To prepare the oral compositions, the agent alone or in combination with other active ingredient(s), may be formulated to yield a dosage of, e.g., from about 0.01 to about 50 mg/kg daily, or from about 0.05 to about 20 mg/kg daily, or from about 0.1 to about 10 mg/kg daily. Oral tablets may include the active ingredient(s) mixed with compatible pharmaceutically acceptable excipients such as diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are exemplary disintegrating agents. Binding agents may include starch and gelatin. The lubricating agent, if present, may be magnesium stearate, stearic acid, or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, active ingredient(s) may be mixed with a solid, semi-solid, or liquid diluent. Soft gelatin capsules may be prepared by mixing the active ingredient with water, an oil, such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions, or syrups, or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The composition used in the present methods can be administered using any suitable delivery mechanisms or techniques. In some embodiments, the composition can be administered alone. In other embodiments, the composition can be administered with a pharmaceutically acceptable carrier or excipient. In some embodiments, the composition used in the present methods, alone or in combination with other active ingredient(s), can be administered via oral, parenteral, rectal, nasal, topical, or ocular routes, or by inhalation. Exemplary parenteral administration can be via intravenous, intramuscular, intraperitoneal, intranasal, or subcutaneous route. In still other embodiments, the composition can be administered via a medicament delivery system or a medical device. Any suitable medicament delivery system or medical device can be used. For example, the medicament delivery system or the medical device can be an implant, e.g., an implant placed during or after bone surgery, a catheter, or a sustained-release drug delivery system.

Sterile compositions are within the present disclosure, including compositions that are in accord with national and local regulations governing such compositions.

The pharmaceutical compositions comprising an agent that alters expression or activity of CHRFAM7A, CHRNA7, α7nAchR, or other human-specific genes (HSGs) or taxonomically-restricted genes (TRGs) in leukocytes, alone or in combination with other active ingredient(s), described herein may further comprise one or more pharmaceutically-acceptable excipients. A pharmaceutically-acceptable excipient is a substance that is non-toxic and otherwise biologically suitable for administration to a subject. Such excipients facilitate administration of the agent, alone or in combination with other active ingredient(s), described herein and are compatible with the active ingredient. Examples of pharmaceutically-acceptable excipients include stabilizers, lubricants, surfactants, diluents, anti-oxidants, binders, coloring agents, bulking agents, emulsifiers, or taste-modifying agents. In preferred embodiments, pharmaceutical compositions according to the various embodiments are sterile compositions. Pharmaceutical compositions may be prepared using compounding techniques known or that become available to those skilled in the art.

The pharmaceutical compositions containing the agent that alters expression or activity of CHRFAM7A, CHRNA7, α7nAchR, or other human-specific genes (HSGs) or taxonomically-restricted genes (TRGs) in leukocytes, alone or in combination with other active ingredient(s), described herein may be formulated as solutions, emulsions, suspensions, or dispersions in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms.

The compositions may be formulated for rectal administration as a suppository. For parenteral use, including intravenous, intramuscular, intraperitoneal, intranasal, or subcutaneous routes, the agent, alone or in combination with other active ingredient(s), may be provided in sterile aqueous solutions or suspensions, buffered to an appropriate pH and isotonicity or in parenterally acceptable oil. Suitable aqueous vehicles can include Ringer's solution and isotonic sodium chloride. Such forms may be presented in unit-dose form such as ampoules or disposable injection devices, in multi-dose forms such as vials from which the appropriate dose may be withdrawn, or in a solid form or pre-concentrate that can be used to prepare an injectable formulation. Illustrative infusion doses range from about 1 to 1000 μg/kg/minute of agent admixed with a pharmaceutical carrier over a period ranging from several minutes to several days.

For nasal, inhaled, or oral administration, the agent, alone or in combination with other active ingredient(s), may be administered using, for example, a spray formulation also containing a suitable carrier.

For topical applications, the agent, alone or in combination with other active ingredient(s), are preferably formulated as creams or ointments or a similar vehicle suitable for topical administration. For topical administration, the agent, alone or in combination with other active ingredient(s), may be mixed with a pharmaceutical carrier at a concentration of about 0.1% to about 10% of drug to vehicle. Another mode of administering the agent, alone or in combination with other active ingredient(s), may utilize a patch formulation to effect transdermal delivery.

Aspects related to the invention are further described in Dang et al. (2015) “CHRFAM7A: A human-specific α7-nicotinic acetylcholine receptor gene shows differential responsiveness of human intestinal epithelial cells to lipopolysaccharide” FASEB J., 9(6):2292-302; Costantini et al. (2015) “A human-specific α7-nicotinic acetylcholine receptor gene in human leukocytes: Identification, Regulation and the consequences of CHRFAM7A expression” Mol. Med 21(1):323-336; Costantini et al. (2015) “The Human-Specific CHRFAM7A gene is a Human Nicotinic α7-Acetylcholine Receptor Gene that Defines a Selectively Human Inflammatory Response in Epithelial Cells” Immunology; and Baird et al. (2015) “Evidence for a Role of Taxonomically-Restricted and Human-Specific Genes like c2orf40TRG and the CHRFAM7A Nicotinic α7-Acetylcholine Receptor Gene in Defining a Selectively Human Inflammatory Response to Injury” Immunology 2015, the entire content of each is incorporated by reference herewith.

In accordance with the invention, there may be employed conventional molecular biology, microbiology, biochemical, gene therapy, and recombinant DNA techniques within the skill of the art. Such techniques are explained fully in the literature. The invention will be further described in the following examples, which do not limit the scope of the methods and compositions of matter described in the claims.

The invention is also described and demonstrated by way of the following examples. However, the use of these and other examples anywhere in the specification is illustrative only and in no way limits the scope and meaning of the invention or of any exemplified term. Likewise, the invention is not limited to any particular embodiments described here. Indeed, many modifications and variations of the invention may be apparent to those skilled in the art upon reading this specification, and such variations can be made without departing from the invention in spirit or in scope. It is, therefore, intended that the invention is to be limited only by the terms of the appended claims which cover all and full scope of such equivalent variations as fall within the true spirit and scope of the invention.

Throughout the specification various citations are referenced, and the entire content of each is hereby incorporated by reference. The following example is provided to describe the invention in more detail. It is intended to illustrate, not to limit the invention.

### Example 1—Evidence for a Role of Taxonomically-Restricted and Human-Specific Genes Like c2orf40TRG and the CHRFAM7A Nicotinic α7-Acetylcholine Receptor Gene in Defining a Selectively Human Inflammatory Response to Injury

Humans successfully diverged from great apes in part as a consequence of genes being added to (e.g. CHRFAM7A), modified in (e.g. c2orf40TRG) and deleted from (e.g. cmah) the primate genome. Unexpectedly however, the 200+ human-specific genes (HSGs) in the human genome and 1,500+ taxonomically-restricted genes (TRGs) in the primate genome are disproportionately represented amongst genes associated with complex disease.

With remarkably little known regarding any HSGs and TRGs expression in human leukocytes, evidence for a role of the c2orf40TRG and the unique CHRFAM7A HSG (a human nicotinic α7-acetylcholine receptor) in defining a selectively human inflammatory response to injury is presented. First, both c2orf40TRG and CHRFAM7A are highly and widely expressed in normal human leukocytes. Bopth c2orf40TRG and CHRFAM7A HSG are readily detectable in leukocyte cell lines and their gene expression is regulated by unique 500 bp sequences in the respective UTRs. These fragments also contain inflammation-dependent transcription factor binding elements. Immunoblotting demonstrates that both open reading frames encode proteins and RNAseq analyses of transduced HL60 and THP1 leukocytes show that their expression regulates gene pathways associated with cell growth and differentiation, and cell adhesion and leukocyte trans-endothelial migration, respectively. Finally, mice with human hematolymphoid systems show that HSGs and primate TRGs can be studied in vivo.

### Example 2—A Human-Specific α7-Nicotinic Acetylcholine Receptor Gene in Human Leukocytes: Identification, Regulation and the Consequences of CHRFAM7A Expression

The human genome contains a taxonomically-restricted gene that encodes an α7-nicotinic acetylcholine receptor (α7nAChR) gene that is uniquely human. This CHRFAM7A gene originally arose with human speciation and its expression alters the ligand tropism of the homopentameric human α7nAChR ligand-gated cell surface ion channel. To understand its possible significance in regulating human inflammation, its expression in human leukocytes and in leukocyte cell lines was investigated using 5′RACE to identify the CHRFAM7A transcript in THP1 cells, comparing its expression to that of the CHRNA7 gene that encodes the α7nAChR, mapping its distinct promoter, and characterizing the effects of CHRFAM7A transgene expression in human THP1 cells. Both CHRFAM7A and CHRNA7 gene expression were detected in human leukocytes and the levels of both mRNAs were shown to be independent and vary widely. Mapping of the 5′UTR responsible for CHRFAM7A gene expression in THP1 leukocytes identified a 1 kb sequence that was responsible for basal gene expression. Forced over-expression of CHRFAM7A in THP1 cells altered their phenotype and modified the expression of genes associated with focal adhesion (e.g. FAK, P13K, Akt, rhoGEF, Elk1, CycD), leukocyte trans-epithelial migration (Nox, ITG, MMPs, PKC) and cancer (kit, kitL, ras, cFos cyclinD1, Frizzled and GPCR). Most surprisingly, CHRFAM7A expression in THP1 cells up-regulated CHRNA7, which lead to increased binding of the specific α7nAChR ligand, bungarotoxin. Taken together, these data establish a biological consequence to CHRFAM7A expression in human leukocytes and support that this human-specific gene can contribute to, and/or gauge, a human-specific response to inflammation.

The presence of a functionally distinct nicotinic acetyl choline receptor (AChR) on human lymphocytes which appeared to have altered ligand binding have been described. Upon sequencing the human α7nAChR gene on chromosome 15q13,14 was found to be structurally similar to that of all other species. At the same time however, the presence of a second, human-specific partially duplicated α7nAChR-like gene that localized 1.6 Mb 5′ upstream from human CHRNA7 was noted. With only 386 amino acids of the α7nAChR channel domain, this new human-specific gene was initially called “dupα7nAChR” and found to encode an amino terminus that originated from a kinase gene on chromosome 3. The ultimate genetic rearrangement, which occurred after the divergence of humans from other primates, created a new, distinct and human-specific open reading frame (ORF) that produces an exclusively human α7nAChR now called, CHRFAM7A. While many species, including human, great apes, mice and rats have orthologs of CHRNA7 that are generated by alternative splicing of their respective CHRNA7 mRNA, none have a distinct CHRFAM7A gene that is part kinase (FAM/ULK4), part functional α7nAChR and uniquely human.

Since its discovery in 1998, CHRFAM7A has largely been the focus of neuroscience and mental health research because historically the α7nAChR was viewed as a neuron-specific, ligand-gated ion channel. More recently however, its detection in normal human leukocytes has gained particular attention because several in vitro studies have shown that CHRFAM7A modifies α7nAChR channel activity and changes ligand tropism. Because α7nAChR activation is closely tied to the inflammatory responses of peripheral tissues, these observations raise the possibility that CHRFAM7A may be particularly relevant to gauging human inflammation. The Tracey laboratories, for example, established that efferent signaling of the vagus nerve acts exclusively via α7nAChR activation in spleen to regulate systemic cytokine responses to infection in mice. Similarly, Costantini and colleagues demonstrated the existence of a similar α7nAChR-dependent regulating the local inflammatory response in tissues. With α7nAChR activation clearly essential to inflammation not to mention vagus nerve responsiveness and leukocyte function, it is therefore critical to understand how a human-specific α7nAChR in human leukocytes might influence human leukocyte function.

CHRFAM7A expression in normal human leukocytes was investigated, expression in human leukocyte cell lines was compared using 5′RACE to identify the specific CHRFAM7A transcript, comparing CHRFAM7A expression to that of CHRNA7, mapping its promoter, and characterizing its effects on the leukocyte gene expression when expressed in THP1 cells. Because newly evolved genes like CHRFAM7A disproportionately segregate with complex human disease, the results point to the possible existence of CHRFAM7A-dependent contributions to a potentially “human-specific” response to inflammation, that may not be present in other species.

### Materials and Methods

Materials:

The plasmid encoding full-length CHRFAM7A variant 1 (NM 139320.1) was purchased from Origene (Rockville, Md.). The plasmid encoding full-length CHRNA7 variant 2 (EX-Z9777-M51) was obtained from GeneCopoeia (Rockville, Md.). The pGL4 promoter-less expression plasmid encoding firefly luciferase was purchased from Promega. All other chemicals and reagents were the products of Sigma (St Louis, Mo.) unless specified otherwise.

Human Peripheral Leukocytes:

Informed consent was obtained from healthy volunteers for the collection of peripheral blood. Volunteers were recruited and enrolled by the University of California San Diego Clinical Translational Research Institute. Venous blood was collected by peripheral venipuncture in BD Vaccutainer® blood collection tubes containing EDTA (BD Biosciences, Franklin Lakes, N.J.) and placed on ice. Red blood cells were lysed using BD Pharm Lyse™ ammonium chloride solution (BD Biosciences) at room temperature for 15 minutes and leukocytes pelleted by centrifugation. Cell pellets were stored at −80° C. until further analyses. The University of California San Diego Institutional Review Board approved the enrollment of participants, consent forms, and specimen collection protocols.

Cell Culture:

All cell lines were originally purchased from ATCC and/or acquired through the UCSD Department of Surgery, Division of Trauma, Burns and Acute Care Surgery Cell Repository. Thawed cells were washed in RPMI culture media containing 10% FCS, the pellet reconstituted in culture media and cells plated into six-well tissue culture plates. All cells were washed 48 hrs later and allowed to grow to 90% confluence and propagated with trypsin digestion as needed. For transduction studies, cells were seeded at 2×10⁶ in 6-well tissue culture plates the day before the experiment. As indicated in each experiment, cells were harvested directly from the culture dishes for total RNA preparation, processed for stable or transient transfection, or treated with 100 ng/ml LPS (CAT# L4391, Sigma) for 3 hours. At the end of incubations, cells were harvested, total RNA isolated and the cDNA generated (see below) used for analyses of gene expression.

Lentiviral Constructs for CHRFAM7A Expression:

The ORF of human CHRFAM7A variant 1 was amplified by PCR from pCMV6-Entry (Cat#: PS100001, Origene) with forward primer, 5′-AGTCCTCGAGATGCAAAAATATTGCATCT-3′ (SEQ ID NO:9) and reverse primer, 5′-ATTCGGATCCTTACGCAAAGTCTTTGGACACGGC-3′ (SEQ ID NO:10). The PCR products were purified and cloned into pLVX-IRES-ZsGreenl. The identity of the plasmid was confirmed by DNA sequencing (Retrogen). Lenti-CHRFAM7A was packaged using Lenti-X™ HTX Packaging System (Cat#631247, Clontech) following instructions from the vendor. After 48 hours, the supernatant was used to transduce THP1 cells.

Flow cytometry and cell sorting of THP1 cells: For flow cytometry analyses, cells were washed and fixed with Cytofix according to the manufacturer's recommendations (BD Biosciences) for 10 minutes on ice. Cells were then incubated with labeled bungarotoxin (BD Biosciences) in FACs buffer (1% BSA in phosphate buffered saline (PBS) containing 0.005% sodium azide) and washed in FACs buffer. Flow cytometry was performed with a Becton Dickinson FACSCalibur and data analysis performed with CellQuestPro software from Becton Dickinson, processed and analyzed using JFlow. To purify GFP expressing THP1 cells, the transduced cells were sorted twice by FACS at the core facilities of the Center for AIDS Research at UCSD, selected for GFP expression and expanded as cell suspensions. Stable expression was monitored weekly for retention of >85% cells expressing GFP as measured by flow cytometry. Cells were propagated in 10% RPMI1640.

RNAseq, Gene Expression and Pathway Analyses:

Total RNA was prepared from transduced and sorted THP1 cells using RNeasy kit (Qiagen) and was quantified using a Nanodrop Spectrophotometer. One μg total RNA was used for RNAseq analyses and performed by contract with the Genomics Core, Cedars-Sinai Medical Center in Los Angeles. Bioinformatic analyses, differential gene expression and pathway analyses were performed by contract with AccuraSciences. For datasets and RNA-seq differential expression (DE) analysis, the BAM files for Vector and CHRFAM7A stable transduced cells were generated by RNA sent to the genome core facilities at Cedars-Sinai Genomics core at Cedars Sinai Medical Center Los Angeles Calif. and were used for differential gene expression analyses and comparisons made between CHRFAM7A and Vector using two methods to define differentially expression genes. DESeq (Anders et al. (2010) “Differential expression analysis for sequence count data” Gen. biol. 11, R106) is one of the few methods suitable with limited replicates (Rapaport et al. (2013) “Comprehensive evaluation of differential gene expression analysis methods for RNA-seq data” Gen. biol. 14, R95) and controls for false positive signals. The Python package HTseq was used to produce the count table and a P-value <0.05 was set as cutoff. In the second method 2, the results of DESeq were overlapped with Cuffdiff (Trapnell et al. (2010) “Transcript assembly and quantification by RNA-Seq reveals unannotated transcripts and isoform switching during cell differentiation” Nat. biotech. 28, 511-515), and a P-value <0.05 was chosen as cutoff. The differentially expressed gene groups defined by both analytical methods were for functional enrichment analysis and GOseq in bioconductor was used for Gene Ontology analysis (Young et al. (2010) “Gene ontology analysis for RNA-seq: accounting for selection bias” Gen. biol. 11, R14) with up- and down-regulated differentially expressed genes respectively. A P-value cutoff of 0.05 was used to choose significant GO terms. The Functional Class Scoring (FCS) method implemented in GSEABase was used for KEGG pathway analyses (Amarzguioui, et al. (2004) “An algorithm for selection of functional siRNA sequences” Biochem. and biophys. Res. Comm's 316, 1050-1058) and a P-value of <0.05 used to define significant pathway categories.

Isolation of RNA from Cultured Cells and Preparation of cDNA for PCR and q-PCR:

Total RNA was prepared from cell lysates using the RNeasy kit (Qiagen, San Diego Calif.) and was quantified using a Nanodrop Spectrophotometer. One μg of the total RNA was reversed transcribed using iScript cDNA synthesis kit (BioRad, San Diego Calif.) in a 20 μl reaction as described by the manufacturer and 1 μl was used for RT-PCR or real-time qPCR analyses.

RT-PCR and Quantitative RT-PCR for CHRFAM7A and CHRNA7: RT-PCR was performed in a 50 μl reaction containing 45 μl PCR blue mix (Invitrogen), 1 μl of each primer (10 μM), 1 μl cDNA, and 2 μl water. The cycling conditions were: 94° C. for 4 minutes followed by 35 cycles of 94° C. for 30 seconds, 60° C. for 30 seconds, and 72° C. for 60 seconds and a final extension at 72° C. for 5 minutes. Ten μl of each PCR products were resolved on a 2% agarose gel and images were acquired using Alpha Innotech imaging system. Real-time qPCR was performed in a 25 μl reaction containing 12.5 μl 2×CYBR Green PCR Master Mix (BioRad), 0.5 μl of each primer (10 μM), 1 μl cDNA, and 10.5 μl water. PCR cycling conditions were: 95° C. for 10 minutes followed by 45 cycles of 94° C. for 25 seconds, 60° C. for 25 seconds, and 72° C. for 40 seconds. Primer efficiency for CHRFAM7a and CHRNA7 were 100% and 94% respectively. Expression of CHRNA7 and CHRFAM7a was normalized to that of GAPDH using ΔΔCt method.

Primers for CHRFAM7a were:
(SEQ ID NO: 11)

| | |
|---|---|
| Sense: | 5′-ATAGCTGCAAACTGCGATA-3′, |

and
(SEQ ID NO: 12)

| | |
|---|---|
| Anti-sense: | 5′-cagcgtacatcgatgtagcag-3′. |

Primers for CHRNA7 were:
(SEQ ID NO: 13)

| | |
|---|---|
| Sense, | 5′-acATGcgctgctcgccggga-3′, |

and
(SEQ ID NO: 14)

| | |
|---|---|
| Anti-sense: | 5′-gattgtagttcttgaccagct-3′. |

Primers for GAPDH were:
(SEQ ID NO: 15)

| | |
|---|---|
| Sense: | 5′- CATGAGAAGTATGACAACAGCCT-3′, |

and
(SEQ ID NO: 16)

| | |
|---|---|
| Anti-sense: | 5′- AGTCCTTCCACGATACCAAAGT-3′. |

5′ RACE and Identification of CHRFAM7A Variant 1:

5′ RACE was performed using SMARTer™ RACE cDNA Amplification Kit (Clontech) following vendor's instructions. Briefly, total RNA was prepared from THP1 cell with RNeasy kit (Qiagen). Three μg total RNA was processed for mRNA using Poly A Spin mRNA Isolation kit (NEB). One fifth of the poly A RNA was reverse transcribed, and the resulting cDNA was amplified sequentially by PCR and nested PCR. Both gene-specific primers (GSP), GSP and nestGSP listed below, hybridize to the 5^{th} exon of human CHRNA7/CHRFAM7A, with nestGSP 5′ to the GSP without overlapping. The nested PCR products were purified and cloned into pDrive (Qiagen). Colonies were sequenced to identify the 5′ initiation sites and the 5′ sequence upstream the 5^{th} exon of CHRNA7/CHRFAM7a.

| (SEQ ID NO: 17) | |
|---|---|
| GSP | (5′-GCAGGTACTGGCAATGCCCAGAAG-3′) |
| (SEQ ID NO: 18) | |
| nestGSP | (5′-TAGTGTGGAATGTGGCGTCAAAGCG-3′) |

Analyses of the CHRFAM7A promoter: The putative CHRFAM7A promoter region spanning from −2363 to +22 relative to the open reading frame ATG start codon was amplified by PCR of genomic DNA isolated from HEK293 cells. The longest fragment was cloned into pGL4 promoter-less luciferase reporter plasmid (Promega) according to the manufacturers specifications and the resulting plasmid, pGL4-CHRFAM7A (˜2400) was confirmed by DNA sequencing and thereafter referred to f2400 to reflect the size of the fragment.

The primers were: Sense: 5′-ATCAGCTAGCTCTAGATAGACAGCATTTTA-3′ (SEQ ID NO:19) containing a NheI restriction site, and Anti-sense: 5′-GCATAGATCTGGTAGATGCAATATTTTTGCAT-3′ (SEQ ID NO:20) containing a BgIII restriction site.

Three serial 5′ deletion promoter constructs of 1800, 1000, and 500 bp were derived by PCR of the f2400 template using the same anti-sense primer described above, with one of three sense primers to obtain: f1800 (5′-ATCAGCTAGCAAGCCTTCATCAGTGGAAAT-3′) (SEQ ID NO:21), f1000 (5′-ATCAGCTAGCGTATGACTCAAGTCCTTGAC-3′) (SEQ ID NO:22), and f500 (5′-ATCAGCTAGC CTTGCTGTATTCTCTAAACTA-3′) (SEQ ID NO:23).

The fragments generated were cloned into the pGL4 vector to create plasmids f1800, f1000, and f500, which were each sequenced to confirm their identity. These plasmids were then transiently transfected into THP1 cells as described below, and luciferase activity was analyzed 30 hours after transfection following the manufacturer's instructions (Promega). Luciferase activity was normalized to protein concentration and the data presented as relative luciferase activity compared to the activity of promoter-less pGL4 transfected cells.

Transfections of THP1 Cells for Promoter Analyses:

THP1 cells, cultured in RPMI1640 supplemented with 1× Glutamax and 1× Penicillin/Streptomycin, were seeded at 5×10⁵ per well in a 24-well plate two hours before transfection. Transient transfection was performed using Lipofectamine 2000 (Invitrogen). Briefly, 2.5 μl of the Lipofectamine 2000 was added into 50 μl OPTI-MEM (Invitrogen), vortexed for 5 seconds, continued to incubate at room temperature for 5 minutes. One μg plasmid diluted into 50 μl OPTI-MEM was added into the above mixture, vortexed for 5 seconds, and continued to incubate at room temperature for 20 minutes. The DNA-complex was then added drop-wise to cells and cells were continued to incubate for 30 hours. In case of LPS stimulation, LPS was added at 100 ng/ml 3 hours before the 30-hour incubation. Cells were washed with PBS and lysed with 100 μl Passive Lysis buffer at room temperature for 30 minutes with shaking. The lysate was spun down and 10 μl of the supernatant was used for luciferase assay on POLARstar Omega plate reader (BMG LABTECH). Luciferase activity normalized to protein concentration was expressed as fold changes over that of pGL4 transfected cells.

### Results

Detection of CHRFAM7A and CHRNA7 Expression in Human Leukocytes:

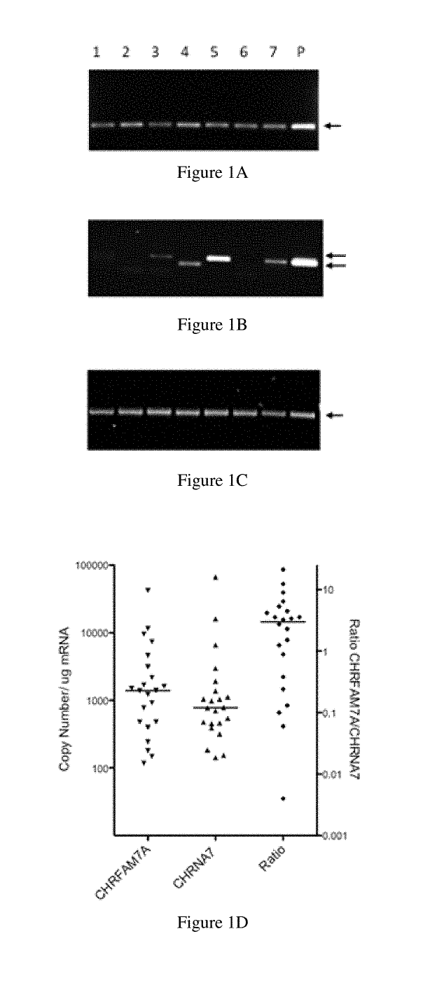

In the course of analyzing gene expression in human leukocytes collected from normal volunteers, the ability to concomitantly detect the expression of CHRNA7 and CHRFAM7A (FIG. 1) was tested. Specific primers were designed to detect CHRFAM7A or both transcripts 1 and 2 of CHRNA7. As shown in FIG. 1A, PCR of leukocyte cDNA prepared from the mRNA seven volunteers established the presence of CHRFAM7A in all samples, although the levels appear to vary (FIG. 1A). Under these same conditions, CHRNA7 was only detected in three of seven samples and of these, two (lanes 4 and 7) had the variant 2 CHRNA7 transcript while the third (lane 5) had variant 1 (FIG. 1B). Because no difference in the signal was obtained in the analyses of GAPDH (FIG. 1C), the results show significant individual variability in expression of both leukocyte CHRNA7 and CHRFAM7A but also suggest that CHRFAM7A, not CHRNA7A is the major form of α7nAChR in human leukocytes. Quantitative analyses showed that CHRNA7A and CHRFAM7A gene expression in human leukocytes (FIG. 1D) were markedly variable and ranged 200-500 fold between different donors N=22).

Identification of the CHRFAM7A Transcript in THP1 Cells:

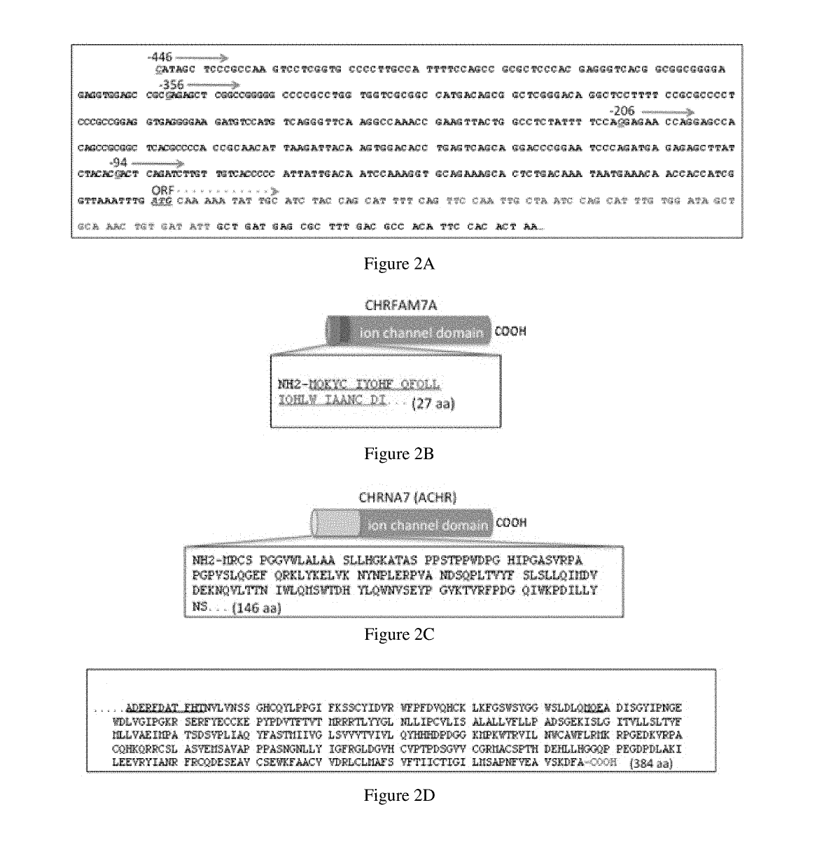

The 5′RACE method was used to extend the CHRFAM7A cDNA clones and amplify the 5′ sequences of the corresponding mRNAs because it only requires the primer to anneal within a known sequence of a cDNA clone. Accordingly, 5′RACE was able to first identify the CHRFAM7A transcript expressed in leukocytes, second deduce the primary sequence of leukocyte CHRFAM7A, third, identify the 5′untranslated region (UTR) sequence responsible for the start of CHRFAM7A transcription and fourth, identify potential promoter elements in the CHRFAM7A 5′UTR (FIGS. 2A-2D). Thermostable DNA polymerase was directed to the CHRFAM7A target RNA by a single primer that was derived from the known CHRFAM7A sequence while the second primer was complementary to a homo-polymeric tail that was added via terminal transferase to the 3′ termini of the CHRFAM7A cDNAs transcribed during the preparation of mRNA (see Materials and Methods above). This synthetic tail provided primer-binding upstream of the unknown 5′ sequence of the target CHRFAM7A mRNA. The products of the amplification reaction were then cloned into the plasmid pDrive vector for sequencing. As shown in FIGS. 2A-2D, transcription of the CHRFAM7A gene in THP1 cells exclusively produced the transcript 1 mRNA and no evidence was found for the CHRFAM7A transcript 2. Of the 14 clones sequenced, 11 originated at 206 bp upstream from the CHRFAM7A open reading frame while 1 each derived from −446, −356 and −94 bp respectively (FIG. 2A) (SEQ ID NO:1). From these sequences, the primary sequence of CHRFAM7A was deduced (FIG. 2B) (SEQ ID NO:2). All the mRNA identified encode the same open reading frame (ORF) that translated to a predicted human-specific and 411 amino acid CHRFAM7A protein that has a unique amino terminal 27 amino acid sequence (FIG. 2B). This is the sequence that originates by rearrangement of the partially duplicated CHRNA7 with the ULK sequence of human chromosome 3. These 27 amino acids substitute for the 146 amino acid of the amino terminus CHRNA7 sequence (FIG. 2C) (SEQ ID NO:3) that localizes to the extracellular domain of CHRNA7A. The remaining 384 amino acids in the carboxyl sequence are 100% identical between CHRNA7 and CHRFAM7A (FIG. 2D) (SEQ ID NO:4). These contain the channel and transmembrane domains of CHRNA7. As predicted by databases leukocyte-CHRFAM7A is a 48 kDa protein that is distinct from the 58 kDa CHRNA7.

CHRFAM7A and CHRNA7 Gene Expression in Human THP1 Cells:

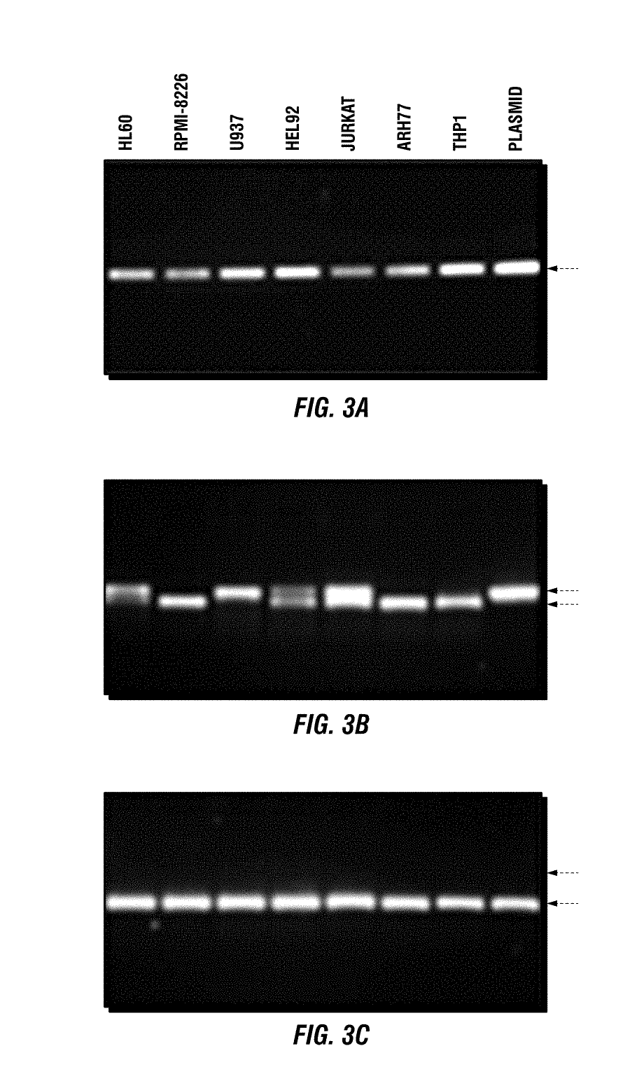

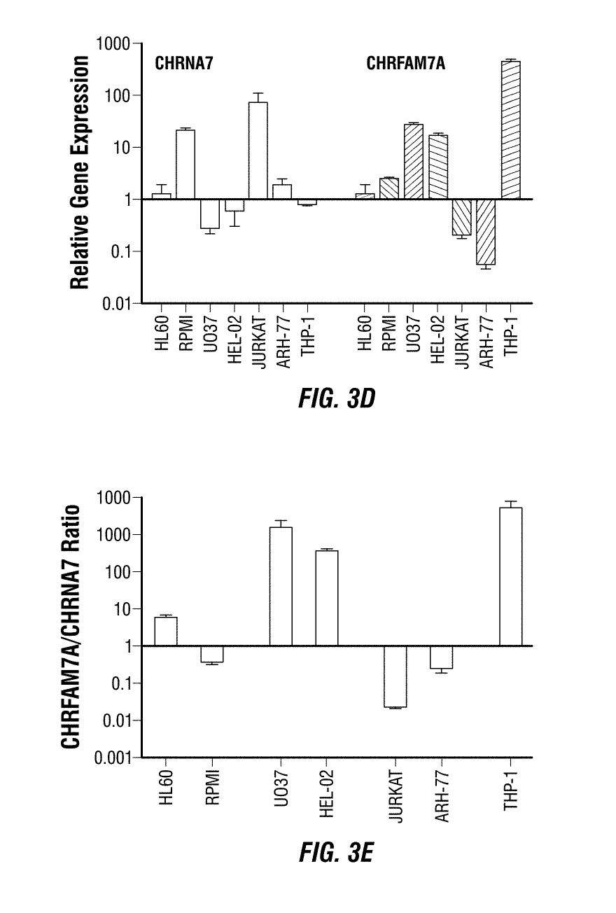

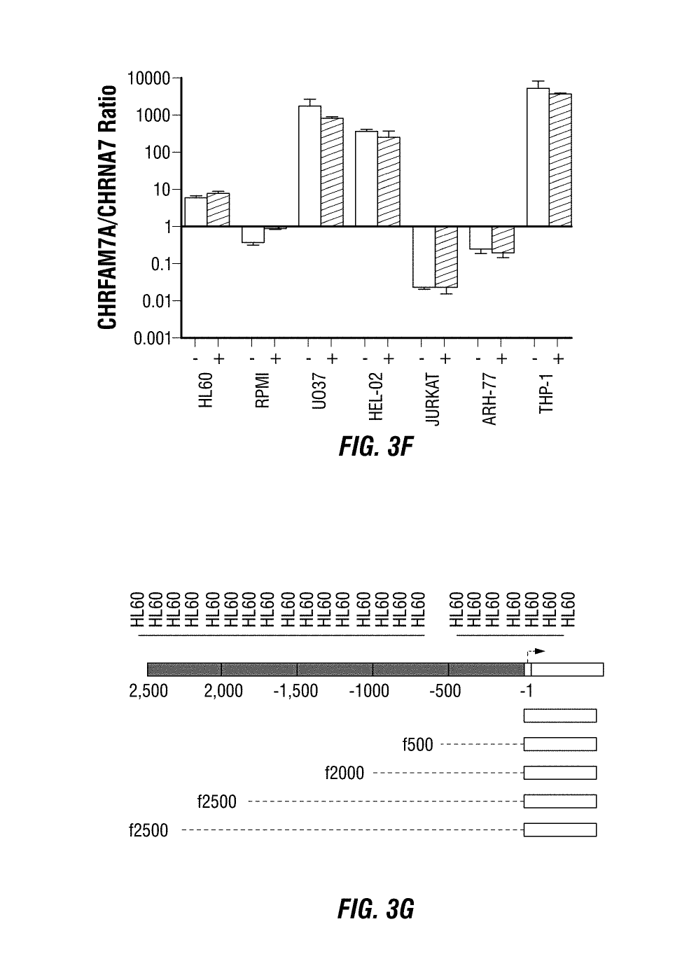

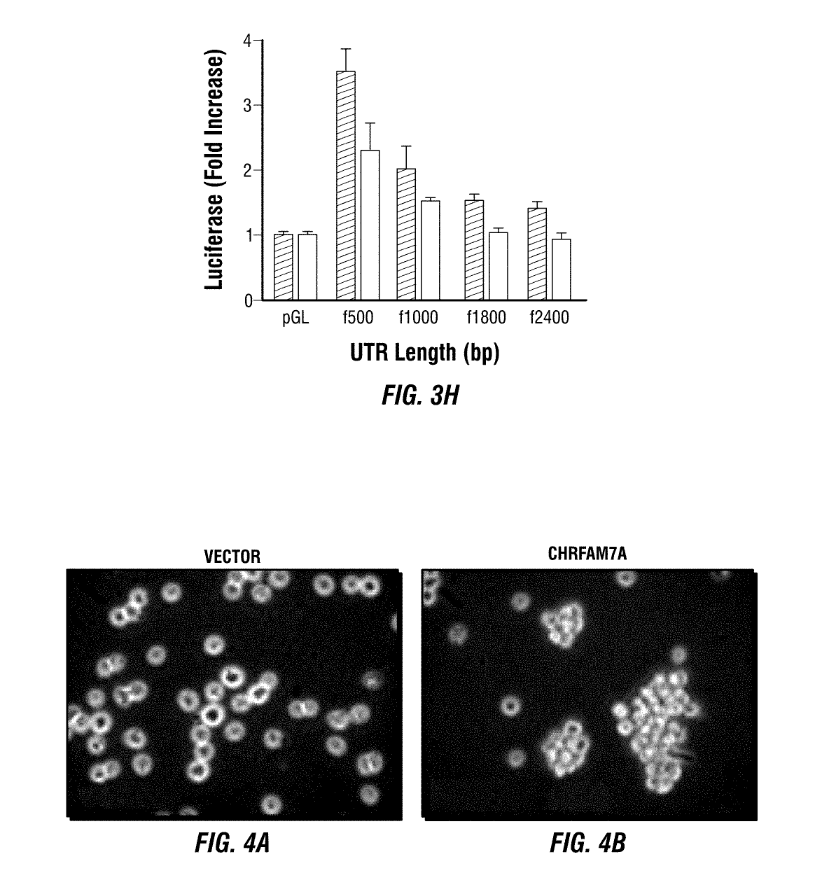

RT-PCR was used to survey the expression of both CHRFAM7A and CHRNA7 in human leukocyte lines (FIGS. 3A-3H). It was found that HL60, RPMI-2286, U937, HEL92, Jurkat, ARH77 are like the pre-monocytic THP1 cell line and express both CHRFAM7A (FIG. 3A) and CHRNA7 (FIG. 3B). They are distinct in two ways however. First they appear to express different levels compared to GAPDH (FIG. 3C) and second, they express different mRNA transcripts that will lead to different α7nAChRs on the cell surface. For example, it was shown that all cells express the CHRFAM7A transcript 1, based on the location of primers and the size of the corresponding amplicon (lane “P” in FIG. 3A). In contrast, three cell lines (HL60, HEL92 and Jurkat) express both transcripts 1 and 2 of CHRNA7 (lane “P” FIG. 3B is transcript 2 of CHRNA7) while U937 cells only express transcript 2 of CHRNA7 and three other cells (RPM-I2286, RH77 and THP1 cells) only express transcript 1 of CHRNA7. This points to significant heterogeneity of the α7nAChR on the human leukocyte cell surface.

These observations were extended using qRT-PCR and quantified the expression of both CHRNA7 (clear bars) and CHRFAM7A (hashed bars) in the different leukocyte cell lines (FIG. 3D). Expression levels were normalized to those detected in HL60 cells and differences in gene expression compared between CHRNA7 and CHRFAM7A. No consistent pattern was observed in the ratio of CHRFAM7A to CHRNA7 (FIG. 3E) which varied 10-10,000 fold higher in some cells (e.g. HL-60, U937, HEL92 and THP1 cells), were near equal in others (RPMI-2286 cells) or 10-100 lower in Jurkat and ARH77 cells. This ratio was also unaffected by incubating cells with LPS (FIG. 3F) implying that LPS affects the expression of both genes equally.

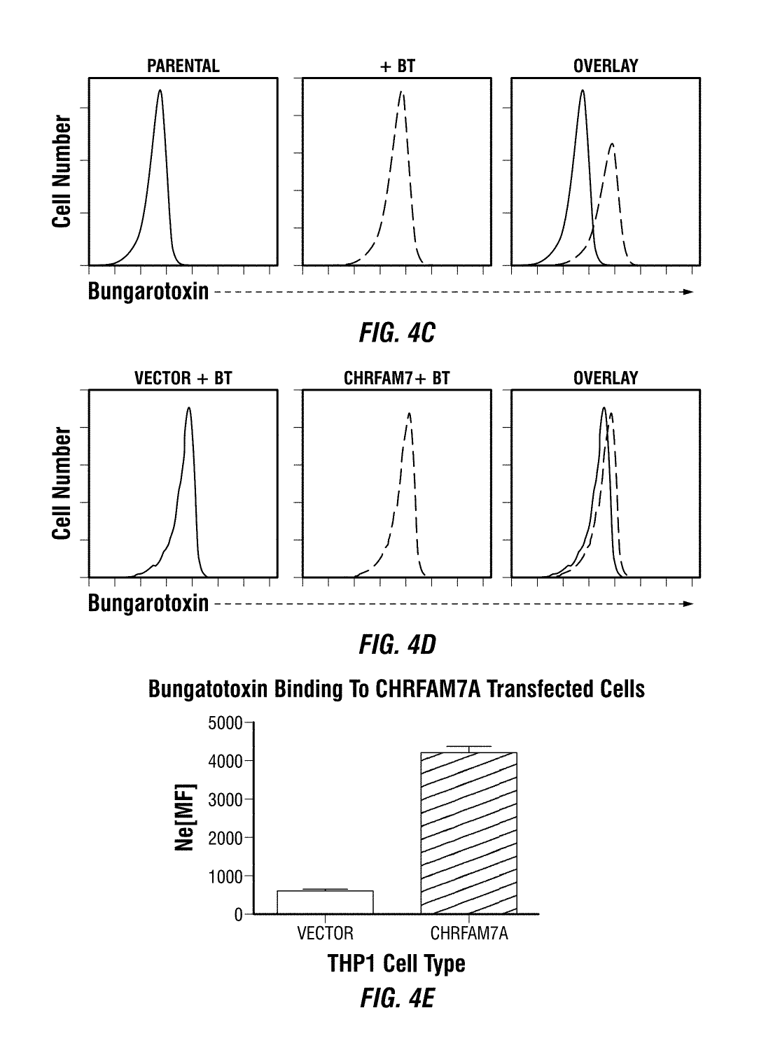

Knowing the 5′UTR sequence and the translation initiation site of the CHRFAM7A gene from the 5′RACE analyses (FIG. 1), a bioinformatic approach was used to identify potential transcription factor binding sites (Cartharius et al. (2005) “MatInspector and beyond: promoter analysis based on transcription factor binding sites” Bioinformatics 21, 2933-2942) and traditional promoter mapping to assess the regulation of CHRFAM7A gene expression. The five 5′UTR constructs contained sequences of +22 to −2400 bp from the CHRFAM7A open reading frame and were prepared as described in the Materials and Methods above. Each plasmid was then tested for its ability to activate luciferase gene expression in THP1 cells (FIG. 3G). Promoter activity was observed within 500 bp of the CHRFAM7A open reading frame. The 5′ extensions of this fragment did not increase luciferase detection but instead decreased luciferase activity pointing to both stimulatory and inhibitory transcriptional elements in the CHRFAM7A promoter. These elements are activated when the same experiment was performed on THP1 cells that pre-treated with 100 ng/ml LPS (FIG. 3H). While a very similar profile in luciferase expression is observed (FIG. 4D), the signal generated by all fragments is decreased consistent with its reported down regulation by LPS.

Biological Consequence of CHRFAM7A Gene Expression in THP1 Cells.

Lentiviral transduced THP1 cells that over-express CHRFAM7A and GFP are distinguishable from control THP1 cells that only express GFP. As shown in FIGS. 4A and 4B, CHRFAM7A transduced cells tend to proliferate as loosely associated cell clusters, which are presumably clonal. This is in contrast to the even distribution of parental and vector transduced THP1 cells and suggest that CHRFAM7A may regulate cell-cell adhesion. Because previous data (FIG. 3A) showed that THP1 cells express both CHRFAM7A and CHRNA7, flow cytometry was used to show that parental THP1 cells bind the specific α7nAChR ligand, bungarotoxin (FIG. 4C). This irreversibly binding ligand toxin is a specific determinant of α7nAChR ligand binding that distinguishes the cell surface channel/receptor from other nicotinic receptors. When bungarotoxin binding to vector and CHRFAM7A transduced cells was compared however (FIG. 4D), a significant increase in bungarotoxin binding in CHRFAM7A transduced cells was detected (FIG. 4E). These data suggested that CHRFAM7A contributed to increased ligand binding either directly by altering ligand binding to a heteropentameric complex or by regulating CHRNA7 gene expression and facilitating not inhibiting, α7nAChR transport to the cell surface.

To assess the effects of CHRFAM7A on basal gene expression in THP1 cells, isolated mRNA from both vector- and CHRFAM7A-transfected cells and their respective transcriptomes by RNA-seq were analyzed. Clustering analyses of gene expression were performed using both DESeq and CutDiff analytical tools. In comparing the effects of CHRFAM7A and GFP-Vector gene expression, 653 differentially expressed genes were identified by DESeq, and 139 differentially expressed genes identified by Cuffdiff. The top 30 up- and down-regulated differentially expressed genes are presented in Table 1 and sorted on the basis of the statistical significance of the change. As expected, the highest differentially expressed gene included CHRFAM7A (55.4 fold) in the CHRFAM7A-transduced cells. It is particularly noteworthy however that an increase in CHRNA7 expression (13.3 fold) was the second most significant difference in CHRFAM7A transfected cells. This suggests that increased bungarotoxin binding in CHRFAM7A cells (FIG. 4D) may be the result of increased α7nAChR on the cell surface rather than a reflection of an increase in a CHRFAM7A subunit. These data support the hypothesis that CHRFAM7A may modulate CHRNA7 availability to the surface perhaps with newly synthesized CHRFAM7A protein that can reportedly form a heteropentamer with modified ligand specificity, tropism and binding kinetics to the α7nAChR homopentamer. Amongst other significantly changed differential genes, Versican (#1, 4.5 fold), Tensin-like protein (#4, 8.7 fold), SIGLEC1 (#7, 3.6 fold), Glipican-6 (#15, 5.1 fold) and EPSTI1 (#19, 3.3 fold) expression all tie to cell adhesion which itself is a phenotypic difference of CHRFAM7A- and vector-transduced cells (FIG. 4). Interestingly, there are also two genes encoding antisense (PAX-AS1) and microRNA with differential expression that is nearly as high (44.3 and 40.6 fold) as the 55 fold change elicited by lentiviral transduction with CHRFAM7A. Finally, it is also interesting to note that six of the most significantly altered genes are tied to interferon including IFI6 (#5), IFI44 (#6), IFIT2 (#11) IF44L (#13), IFIT1 (#14) AND IFI27 (#19).

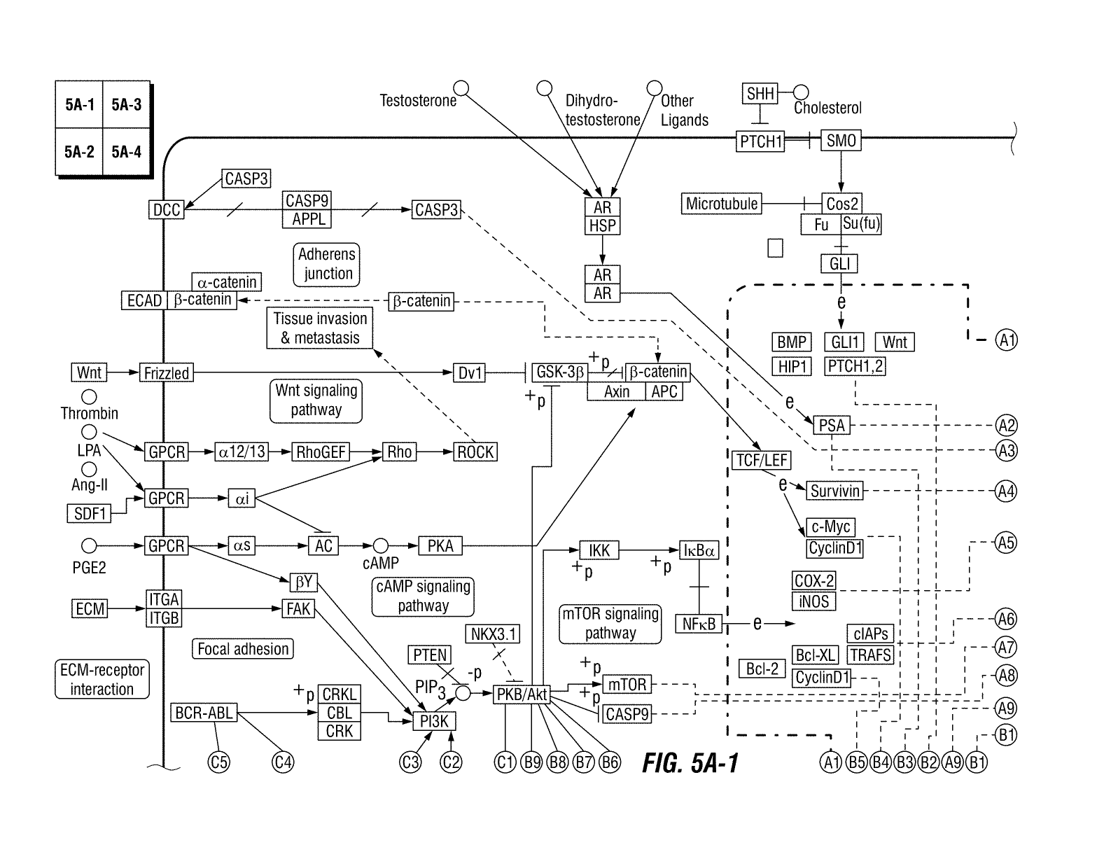

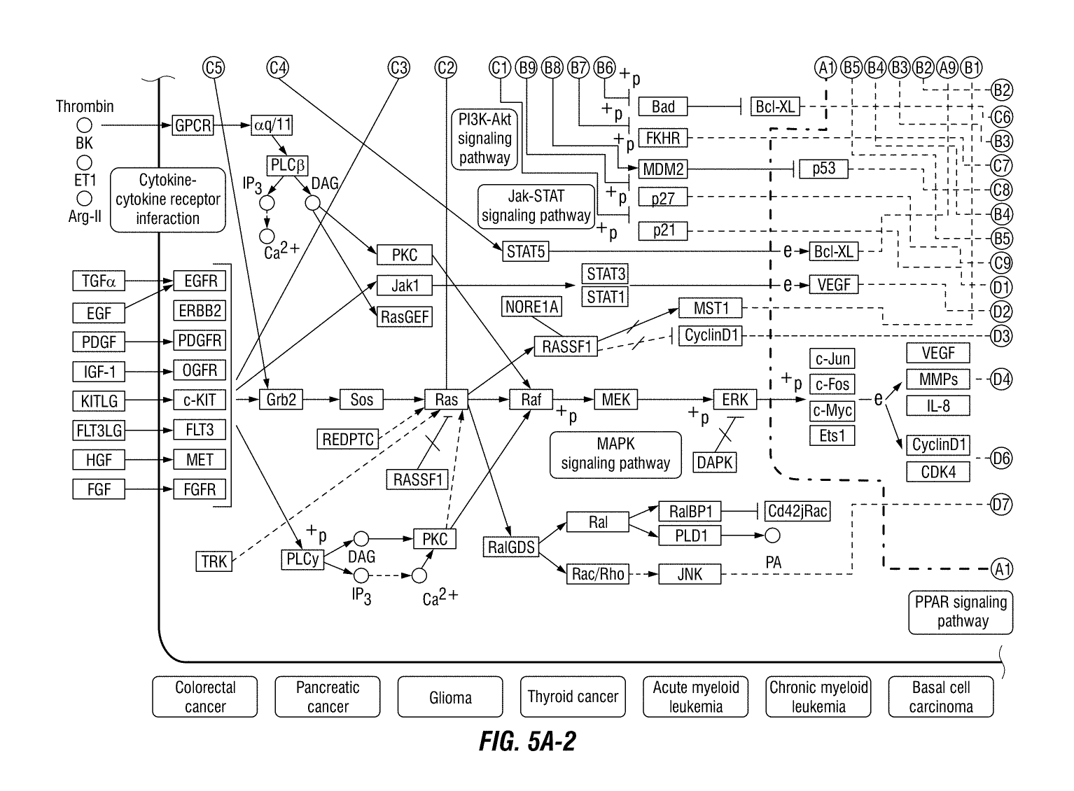

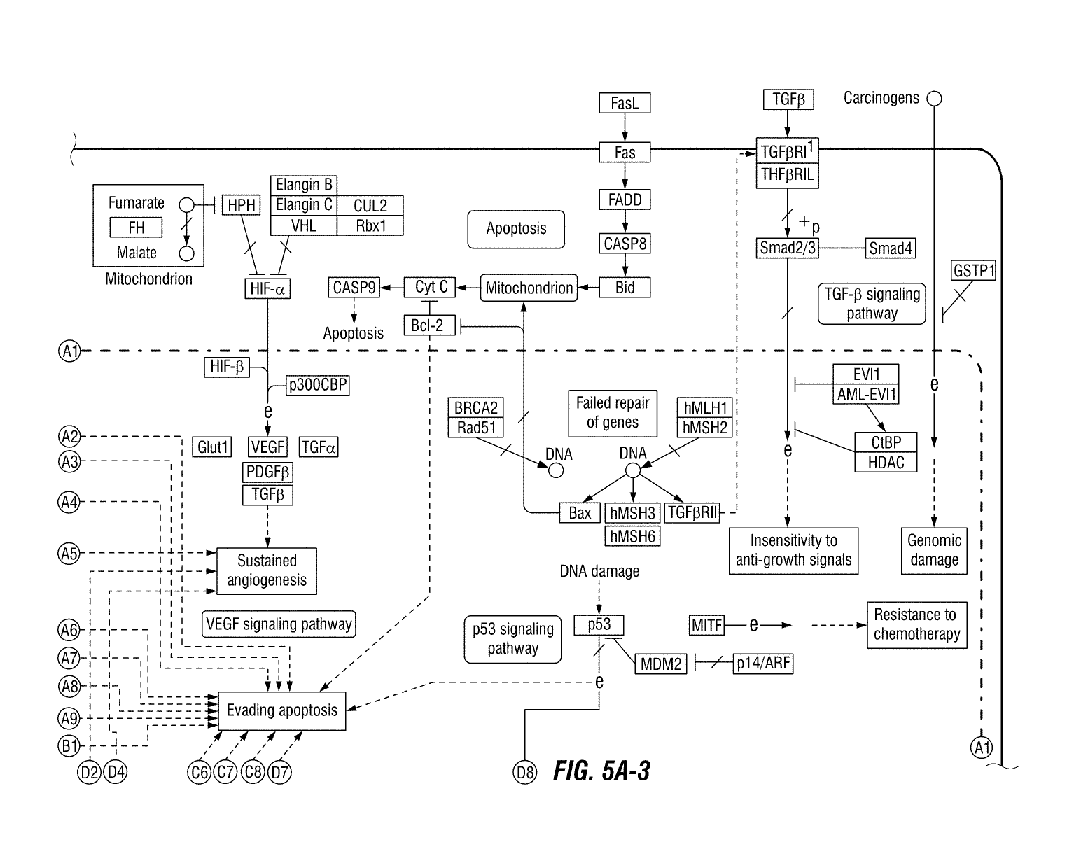

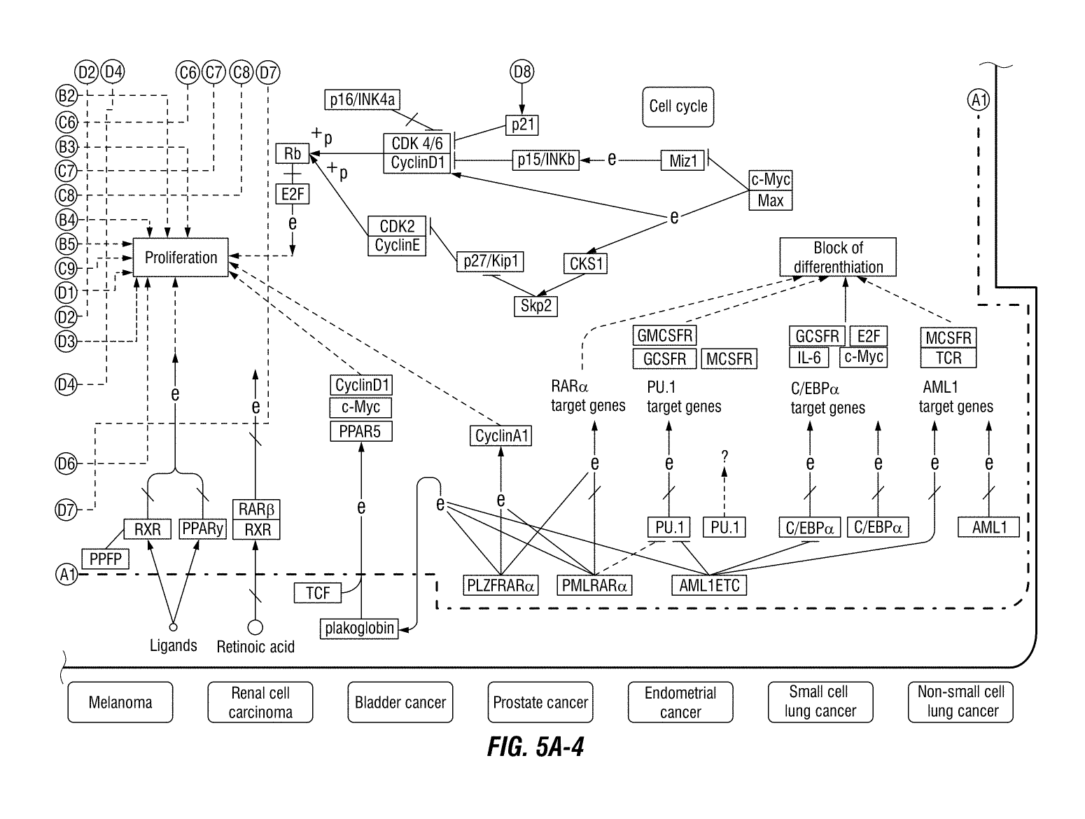

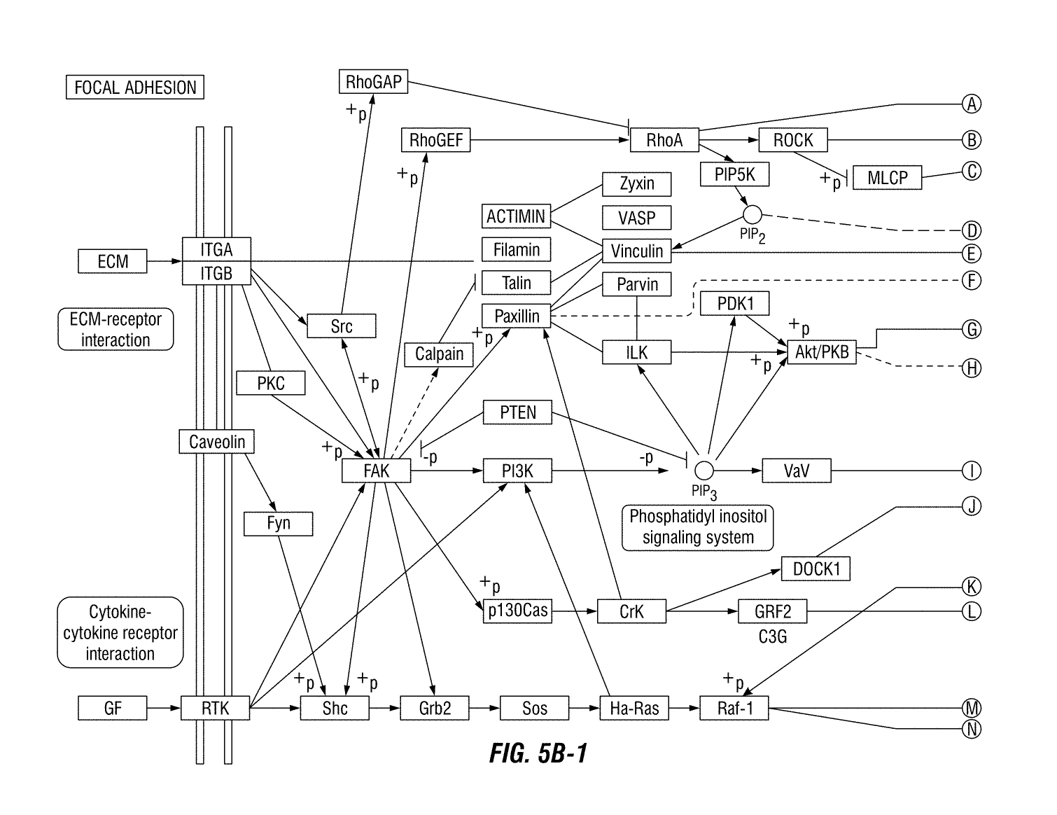

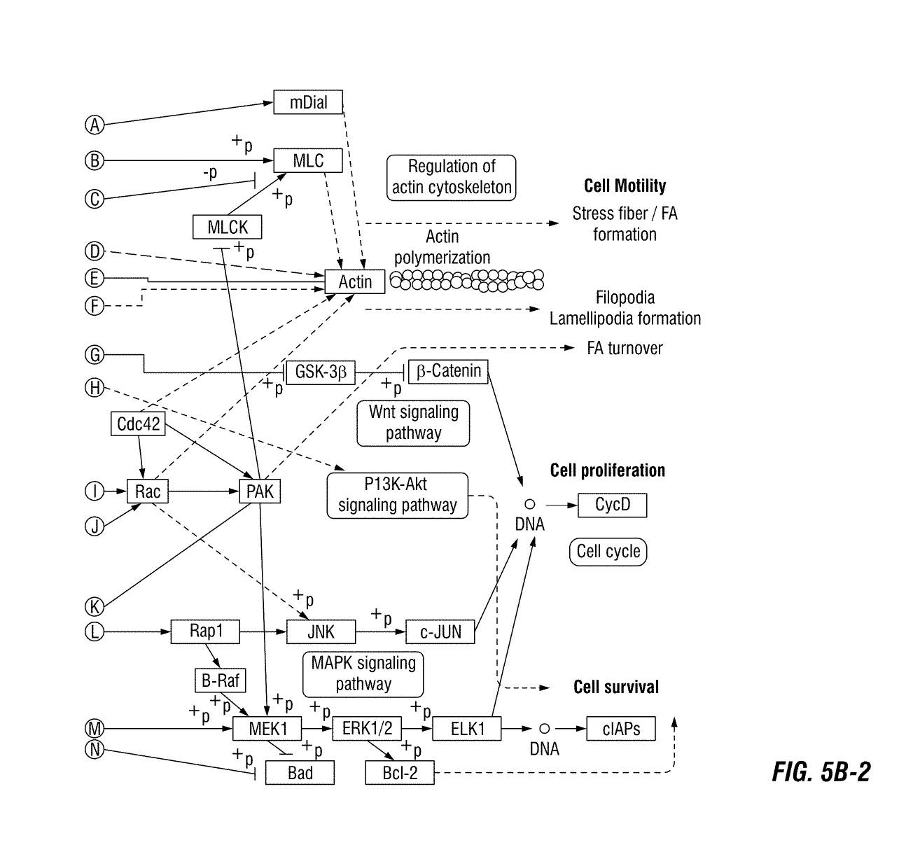

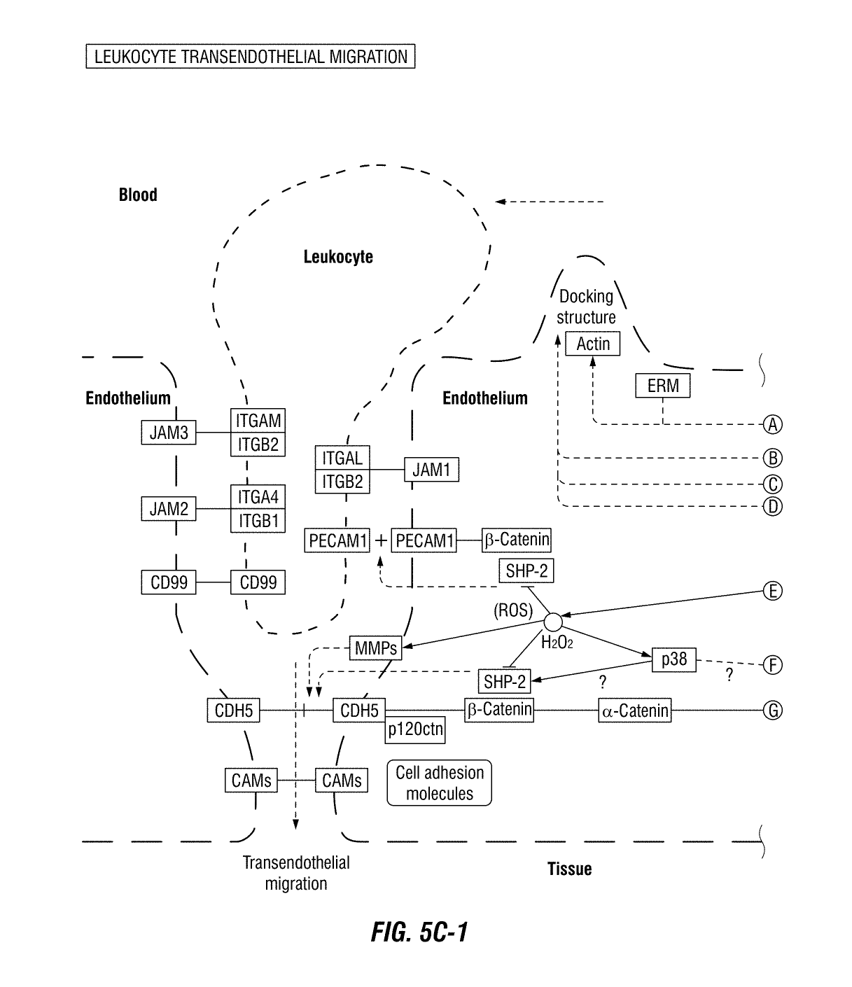

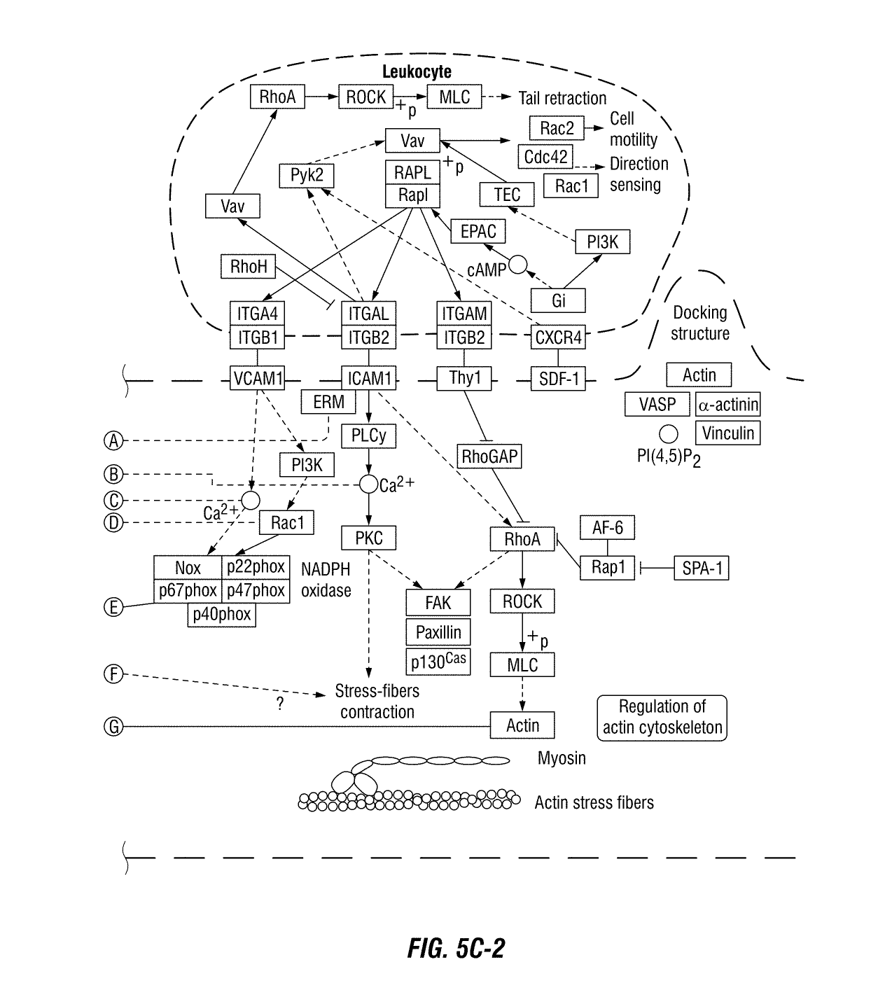

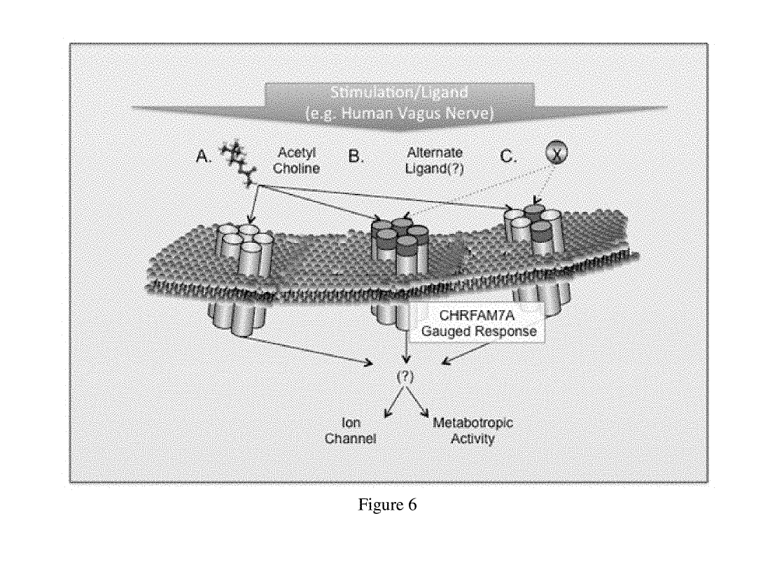

In a test to analyze the effects of CHRFAM7A expression on differential gene expression, GO enrichment analyses of CHRFAM7A-induced changes were analyzed in biological process, cellular components and molecular functions. As shown in Table 2, the top five most significantly enriched GO terms in each of the up- and down-regulated differentially expressed genes included the Type 1 interferon pathway, cell responses to interferon and cell adhesion. In another test, Kegg pathways most affected of differential gene expression were evaluated in each of the differentially expressed groups (Table 3). Their contribution to known pathways of cancer, leukocyte trans-endothelial migration and focal adhesion are presented (FIG. 5).

TABLE 1
Top Differentially Up and Down Regulated Genes, Sorted by Significance of Change.

| | A. ENSMBL | Entrez | I.D. | Gene Name | Δ | p |
|---|---|---|---|---|---|---|
| 1 | ENSG00000038427 | 1462 | VCAN | Veriscan | 4.5 | 7.40E−17 |
| 2 | ENSG00000175344 | 1139 | CHRNA7 | Cholinergic receptor, nicotinic, alpha 7 (neuronal) | 13.3 | 2.50E−15 |
| 3 | ENSG00000189223 | 654433 | PAX8-AS1 | PAX8 antisense RNA 1 | 44.3 | 2.80E−12 |
| 4 | ENSG00000100181 | 387590 | TPTEP1 | Transmembrane Phosphatase Tensin Homology | 6.7 | 8.20E−12 |
| 5 | ENSG00000126709 | 2537 | IFI6 | Interferon, alpha-inducible protein 6 | 3.5 | 2.00E−11 |
| 6 | ENSG00000137965 | 10581 | IFI44 | Interferon-induced protein 44 | 6.7 | 1.50E−09 |
| 7 | ENSG00000088827 | 6614 | SIGLEC1 | Sialic acid binding Ig-like lectin 1, sialoadhesin | 3.6 | 3.80E−09 |
| 8 | ENSG00000166664 | 89632 | CHRFAM7A | Transfected Gene | 55.4 | 7.80E−09 |
| 9 | ENSG00000166104 | 102466227 | MIR7162 | Micro RNA 7162 | 40.6 | 9.50E−07 |
| 10 | ENSG00000105666 | 51477 | ISYNA1 | Inositol-3-phosphate synthase 1 | 9.1 | 1.70E−06 |
| 11 | ENSG00000119922 | 3433 | IFIT2 | Interferon-induced protein tetratricopeptide repeats 2 | 3.6 | 2.20E−06 |
| 12 | ENSG00000206337 | 10866 | HCP5 | HLA complex P5 | 12 | 6.40E−06 |
| 13 | ENSG00000137959 | 10964 | IFI44L | Interferon-induced protein 44-like | 4.9 | 4.00E−05 |
| 14 | ENSG00000185745 | 3434 | IFIT1 | Interferon-induced protein tetratricopeptide repeats 1 | 3.9 | 5.20E−05 |
| 15 | ENSG00000183098 | 10082 | GPC6 | Glypican 6 | 5.1 | 6.30E−06 |
| 16 | ENSG00000170365 | 4086 | SMAD1 | SMAD family member 1 | 3.8 | 8.90E−05 |
| 17 | ENSG00000179796 | 116135 | LRRC3B | Leucine rich repeat containing 3B | 4.6 | 0.0002 |
| 18 | ENSG00000133106 | 94240 | EPSTI1 | Epithelial stromal interaction 1 | 3.3 | 0.0002 |
| 19 | ENSG00000165949 | 3429 | IFI27 | Interferon-induced protein 27 | 6.1 | 0.0006 |
| 20 | ENSG00000081923 | 5205 | ATP8B1 | ATPase, aminophospholipidtransporter, type 8b member 1 | 0.31 | 0.0008 |
| | B. ENSMBL | Entrez | Gene | Gene Name | Δ | p |
| 21 | ENSG00000174099 | 253827 | MSRB3 | Methionine sulfoxide reductase B3 | 0.37 | 1.80E−07 |
| 22 | ENSG00000156515 | 3098 | HK1 | Hexokinase 1 | 0.42 | 8.60E−07 |
| 23 | ENSG00000100060 | 4242 | MFNG | MFNG O-fucosyl 3-beta-N-acetylglucos-aminyl-transferase | 0.3 | 1.30E−06 |
| 24 | ENSG00000100234 | 7078 | TIMP3 | TIMP metallopeptidase inhibitor 3 | 0.51 | 1.40E−06 |
| 25 | ENSG00000182263 | 55137 | FIGN | Fidgetin | 0.08 | 3.30E−06 |
| 26 | ENSG00000165629 | 1602 | DACH1 | Dachshund family transcription factor 1 | 0.46 | 1.50E−05 |
| 27 | ENSG00000134824 | 9415 | FADS2 | Fatty acid desalurase 2 | 0.58 | 0.0002 |
| 28 | ENSG00000126767 | 2002 | ELK1 | ELK1, member of ETS oncogene family | 0.42 | 0.0002 |
| 29 | ENSG00000144712 | 23066 | CAND2 | Cullin-associated and neddylation-dissociated 2 | 0.31 | 0.0006 |
| 30 | ENSG00000011600 | 7305 | TYROBP | TYRO protein tyrosine kinase binding protein | 0.59 | 0.0007 |

TABLE 2
Top Five Significantly Enriched (Up- and Down-Regulated) GO Terms in Differentially
Expressed Genes

| | | | False |
|---|---|---|---|
| | | | Discovery |
| | Enriched GO terms (for DE genes by method 1) | P-value | Rate |
| Up Regulated by | Type I interferon signaling pathway | 6.93E−019 | 5.23E−015 |
| CHRFAM7A | Cellular response to type I interferon | 6.93E−019 | 5.23E−015 |
| (DESeq) | Response to type I interferon | 8.79E−019 | 5.23E−015 |
| | Response to stimulus | 7.83E−017 | 2.28E−013 |
| | Defense response to virus | 8.82E−017 | 2.28E−013 |
| Up Regulated by | Response to type I interferon | 0.00E+000 | 0.00E+000 |
| CHRFAM7A | Type I interferon signaling pathway | 0.00E+000 | 0.00E+000 |
| (Cuffdiff) | Cellular response to type I interferon | 0.00E+000 | 0.00E+000 |
| | Response to chemical | 1.44E−011 | 6.15E−008 |
| | Response to organic substance | 1.72E−011 | 6.15E−008 |
| Down Regulated | Molecular_function | 7.90E−011 | 1.41E−006 |
| by | Binding | 1.61E−010 | 1.44E−006 |
| CHRFAM7A | Cytosolic ribosome | 8.17E−010 | 2.82E−006 |
| (DESeq) | Cytosolic large ribosomal subunit | 9.47E−010 | 2.82E−006 |
| | Biological_process | 9.49E−010 | 2.82E−006 |
| Down Regulated | Calcium-dependent cell-cell adhesion | 3.40E−005 | 1.59E−001 |
| by | Extracellular region | 3.41E−005 | 1.59E−001 |
| CHRFAM7A | Cell adhesion | 3.54E−005 | 1.59E−001 |
| (Cuffdiff) | Biological adhesion | 3.57E−005 | 1.59E−001 |
| | Establishment of protein localization to membrane | 1.07E−004 | 3.83E−001 |

TABLE 3
Top Five Most Significantly Enriched KEGG Pathways

| Sample | | | False |
|---|---|---|---|
| Comparison | Enriched Pathways | P-value | Discovery Rate |
| CHRFAM7A | Ribosome | 3.35E−003 | 4.40E−001 |
| (DESeq) | Pathways in cancer | 5.68E−003 | 4.40E−001 |
| | Hepatitis C | 7.37E−003 | 4.40E−001 |
| | Colorectal cancer | 1.16E−002 | 5.20E−001 |
| | Leukocyte trans- | 1.78E−002 | 6.06E−001 |
| | endothelial migration | | |
| CHRFAM7A | Cytokine-cytokine | 1.64E−002 | 4.05E−001 |
| (Cuffdiff) | receptor interaction | | |
| | Osteoclast differentiation | 1.74E−002 | 4.05E−001 |
| | RIG-I-like receptor | 1.78E−002 | 4.05E−001 |
| | signaling pathway | | |
| | Insulin signaling pathway | 2.23E−002 | 4.05E−001 |
| | TGF-beta signaling | 2.76E−002 | 4.05E−001 |
| | pathway | | |

### Discussion

The data presented here establish that stable over-expression of CHRFAM7A gene expression in THP1 cells, a widely used cell model to study human monocytes (Qin (2012) “The use of THP-1 cells as a model for mimicking the function and regulation of monocytes and macrophages in the vasculature” Atherosclerosis 221, 2-11), has functional effects on basal gene expression. It is also shown that CHRFAM7A is normally expressed in human leukocytes (FIGS. 1 and 2) which, in view of its capacity to gauge α7nAChR activity after transient transfection in vitro, implies that CHRFAM7A has the potential to modulate human leukocyte function and presumably the α7nAChR regulation of inflammation. Interestingly, it was found that the stable transduction of CHRFAM7A also increased basal expression of CHRNA7 thereby establishing the existence of a concomitant and compensatory response to the human specific gene. These data point to the possibility that the ratio of CHRFAM7A and CHRNA7 expression is important and that they are co-regulated. That being said, the identity of the CHRFAM7A sequence was established, the amino acid difference that distinguishes the human CHRFAM7A protein from α7nAChR was deduced and its unique promoter was mapped to a 5′UTR sequence −500 to −1000 bp from the CHRFAM7A open reading frame (FIGS. 2A-2D). Finally, it was shown that when CHRFAM7A is expressed in THP1 cells, it is biologically active and that differentially expressed genes contribute to several pathways of cell function including cell adhesion, growth and trafficking. In as much as there are no analogous or independently regulated CHRFAM7A-like genes in the genomes of other species, these findings implicate the existence of a human-specific mechanism in human leukocytes to gauge the human inflammatory response.

The studies presented are the first to establish a clear and unambiguous functional consequence to CHRFAM7A gene expression in human leukocytes. RNAseq of transduced cells demonstrated increased CHRNA7 gene expression and increased bungarotoxin binding and established a functional linkage between CHRFAM7A and the α7nAChR protein encoded by CHRNA7. Pathway analyses further suggest that CHRFAM7A has functional effects on leukocytes, namely in adhesion and leukocyte trafficking. These will have to be compared to those of CHRNA7 in this same model system.

Together these data provide compelling evidence supporting that CHRFAM7A plays a role in human leukocyte cell biology, at a minimum by regulating human α7nAChR. For example, CHRFAM7A has the capacity to form cell surface hetero-polymers with the wild type α7nAChR and is reported in some models to either exert a dominant negative effect on α7nAChR, regulate the appearance of α7nAChR on the cell surface, or alter ligand tropism. In as much as a role for α7nAChRs in leukocyte homeostasis is unequivocal, CHRFAM7A might then confer a “human-specific” responsiveness to trophic stimuli.

### Example 3—The Human-Specific CHRFAM7A Gene is a Human Nicotinic α7-Acetylcholine Receptor Gene that Defines a Selectively Human Inflammatory Response in Epithelial Cells

Newly evolved genes are disproportionately represented amongst genes associated with complex disease but remarkably little is known regarding their expression, physiological function or the possibility that they can confer species-selectivity to biological responses. CHRFAM7A gene is a case in point. Emerging in the human genome after human speciation from primates, CHRFAM7A encodes a unique α7-nicotinic acetylcholine receptor (α7nAChR) that, when expressed, is a species-specific dominant negative regulator of the ligand-gated α7nAChR ion channel. By using a combination of immunoblotting, RT-PCR, quantitative PCR, molecular cloning and promoter analyses to demonstrate that CHRFAM7A expression can be tied to the human epithelial inflammatory response to injury. Immunoblotting demonstrates that the CHRFAM7A ORF encodes an α7nAChR-like protein. RT-PCR shows that CHRFAM7A mRNA is widely expressed in intestinal epithelial cell lines. CHRFAM7A is also differentially expressed (when compared to α7nAChR) in colon epithelial (e.g. FHs-INT) cells incubated with LPS (100 ng/ml). This is likely attributed to a promoter identified in a 500 bp sequence that contains inflammation-dependent transcription factor binding elements. As CHRFAM7A expression is reported to modulate nicotine binding to, and alter the activity of the α7nAChR, these findings point to the existence of a species-specific α7nAChR response that regulate gut epithelial function in a human-specific fashion.

### Example 4—CHRFAM7A: A Human-Specific α7-Nicotinic Acetylcholine Receptor Gene Shows Differential Responsiveness of Human Intestinal Epithelial Cells to Lipopolysaccharide

The human genome contains a unique, distinct and human-specific α7-nicotinic acetylcholine receptor (α7-nAChR) gene (CHRNA7) called CHRFAM7A on a locus of chromosome 15 associated with mental illness, including schizophrenia. Located 5′ upstream from the “wild type” CHRNA7 gene that is found in other vertebrates, CHRFAM7A expression in a broad range of epithelial cells was demonstrated and the CHRFAM7A transcript found in normal human fetal small intestine epithelial (FHs) cells was sequenced to prove its identity. CHRFAM7A expression was compared to CHRNA7 in eleven gut epithelial cell lines, showing that there is a differential response to lipopolysaccharide when compared to CHRNA7, and the CHRFAM7A promoter was characterized. CHRFAM7A and CHRNA7 gene expression are widely distributed in human epithelial cell lines but the levels of CHRFAM7A gene expression vary up to 5,000-fold between different gut epithelial cells. A 3 hour treatment of epithelial cells with 100 ng/ml lipopolysaccharide (LPS) increased CHRFAM7A gene expression by almost 1000-fold but had little to no effect on CHRNA7 gene expression. Mapping the regulatory elements responsible for CHRFAM7A gene expression identifies a 1 kb sequence in the UTR of the CHRFAM7A gene that is modulated by LPS. Taken together, these data establish the presence, identity and differential regulation of the human-specific CHRFAM7A gene in human gut epithelial cells. In light of the fact that CHRFAM7A expression is reported to modulate ligand binding to, and alter the activity of the wild type α7-nAChR ligand-gated pentameric ion channel, the findings point to the existence of a species-specific α7-nAChR response that might regulate gut epithelial function in a human-specific fashion.

Although the α7-nicotinic acetylcholine receptor protein (α7-nAChR) was originally identified as a neuronal homopentameric ligand-gated ion channel, numerous studies have established that its gene, CHRNA7, is widely expressed in non-neuronal cell types including monocytes, endothelial and epithelial cells and even in various cancer cells where it can regulate inflammation, cell growth and differentiated cell function. Accordingly, it is not surprising that there is significant α7-nAChR in, and out, of the central nervous system, including in the capillary and aortic vasculature, bronchial and small airway epithelium and, in gut, skin and oral epithelial cells and keratinocytes.

With these findings, there has been commensurate interest in defining the biological role of α7-nAChR in peripheral tissues, and most notably the possibility that it functions in cell-cell communication, epithelial barrier integrity, regulating inflammation and/or controlling differentiated function. In this capacity, intestinal epithelial cells are particularly relevant because they serve as critical regulators of barrier function and immune homeostasis. To this end, several studies have implicated α7-nAChR with the proliferation, migration and invasion in various epithelial cells and in the mechanism of nicotine-dependent cell transformation. For example, nicotine treatment of cells can increase growth factors (e.g. VEGFs, HGF, TGFβ, TGFα and PDGFs), their receptors (e.g. VEGFR2, HGFR, EGFR and PDGFR), signal transduction pathways (e.g. MAP kinase, Raf-1, ERK1/2 and MEK1) and, transcription factors (e.g. HIF1α, GATA3, NFκB and STAT-1) in epithelial cells. In this capacity, the α7-nAChR can act as a ligand-gated ion channel or stimulate intrinsic signal transduction and metabotropic activities.

In view of the significance of α7nAChRs to epithelial biology and the observation that human-specific genes are disproportionately implicated in complex disease, it is remarkable that little attention has been paid to the 1998 discovery that there exists a human-specific gene called CHRFAM7A, that can modify α7nAChR responsiveness. Several investigators have associated CHRFAM7A expression in the central nervous system with mental illness but it has also been detected expression in human leukocytes. To date however, there are no reports describing the expression of CHRFAM7A in human epithelial cells. This, and the fact that the expression of CHRFAM7A modulates the biological response to α7nAChR activation has led to the hypothesis that there might be differential CHRFAM7A expression in the human gut. Underscored by the observation that taxonomical studies have described how newly evolved genes are more likely to be associated with complex disease than old genes, it was investigated whether epithelial cells express CHRFAM7A, the CHRFAM7A transcript found in gut epithelial cells was identified, and its expression compared to that of CHRNA7. In analyzing the regulation of CHRFAM7A gene expression, it was found that a differential response to lipopolysaccharide (LPS) that alters the ratio of CHRFAM7A to CHRNA7 in epithelial cells and as such, may point to the existence of human-specific α7nAChR responses in the human gut epithelium.

### Materials and Methods

Materials:

The plasmid encoding full-length CHRFAM7A variant 1 (RC215588) with a DDK-tag sequence at its Carboxyl terminus was purchased from Origene (Rockville, Md.). The plasmid encoding full-length CHRNA7 variant 2 (EX-Z9777-M51) was obtained from GeneCopoeia (Rockville, Md.). The pGL4 expression promoter-less reporter plasmid encoding firefly luciferase was purchased from Promega. The anti-DDK monoclonal antibody (TA50011-100) used in immunoblotting was purchased from Origene. All other chemicals and reagents were the products of Sigma (St Louis, Mo.) unless specified otherwise.

Cell Culture:

All epithelial cancer cell lines were originally purchased from American Type Culture Collection and propagated as instructed. Normal human small intestine epithelial cells (FHs-Int-74) were also obtained from the ATCC (CCL-241). Cells were seeded at 2×10⁶ in 6-well tissue culture plates the day before the experiment. As indicated, cells were either harvested directly for total RNA preparation, processed for transient transfection, or treated with LPS (CAT# L4391, Sigma) at 100 ng/ml for 3 hours. At the end of the incubation with LPS, cells were harvested and used for analyses of gene expression.

Isolation of RNA from Cultured Cells and Preparation of cDNA for PCR and q-PCR:

Total RNA was prepared using RNeasy kit (Qiagen) and was quantitated using Nanodrop Spectrophotometer. One μg total RNA was reverse transcribed using iScript cDNA synthesis kit (BioRad) in a 20 μl reaction. Of the 20 μl cDNA, one μl was used for RT-PCR or real-time qPCR.

PCR and Primers and Conditions for CHRFAM7A and CHRNA7.

RT-PCR was performed in a 50 μl reaction containing 45 μl PCR blue mix (Invitrogen), 1 μl of each primer (10 μM), 1 μl cDNA, and 2 μl water. The cycling conditions were: 94° C. for 4 minutes followed by 35 cycles of 94° C. for 30 seconds, 60° C. for 30 seconds, and 72° C. for 60 seconds and a final extension at 72° C. for 5 minutes. Ten μl of each PCR products were resolved on a 2% agarose gel and images were acquired using Alpha Innotech imaging system. Real-time qPCR was performed in a 25 μl reaction containing 12.5 μl 2×CYBR Green PCR Master Mix (BioRad), 0.5 μl of each primer (10 μM), 1 μl cDNA, and 10.5 μl water. PCR cycling conditions were: 95° C. for 10 minutes followed by 45 cycles of 94° C. for 25 seconds, 60° C. for 25 seconds, and 72° C. for 40 seconds. Primer efficiency for CHRFAM7A and CHRNA7 were 100% and 94% respectively. Expression of CHRNA7 and CHRFAM7A was normalized to that of GAPDH using ΔΔCt method.

Primers for CHRFAM7A were designed to hybridize with the variant 1 transcript by selecting sequences that bridge CHRFAM7A and CHRNA7 and therefore unique to CHRFAM7A and not available in FAM7A or CHRNA7A alone: Sense: 5′-ATAGCTGCAAACTGCGATA-3′ (SEQ ID NO:11), Anti-sense: 5′-cagcgtacatcgatgtagcag-3′(SEQ ID NO:12).

Primers for CHRNA7 were designed to hybridize with both variant 1 and 2 transcripts of CHRNA7 by selecting sequences present in CHRNA7 but absent from CHRFAM7A for amplification: Sense, 5′-acATGcgctgctcgccggga-3′ (SEQ ID NO:13), Anti-sense, 5′-gattgtagttcttgaccagct-3′ (SEQ ID NO:14).

| Primers for human GAPDH were: | |
|---|---|
| (SEQ ID NO: 15) | |
| Sense: | 5′-CATGAGAAGTATGACAACAGCCT-3′, |
| (SEQ ID NO: 16) | |
| Anti-sense: | 5′-AGTCCTTCCACGATACCAAAGT-3′. |

Cloning and Sequencing of Epithelial CHRFAM7A:

To clone and sequence CHRFAM7A, FHs cells were used. FHs cells are an epithelial cell line from normal human small intestine (ATCC CCL-241) which respond like tumor-derived epithelial cells. Cells were seeded at 2×10⁶ per well in a 6-well plate the day before. On the second day, total RNA was extracted using RNeasy kit (Qiagen). One μg total RNA was reverse-transcribed in a 20 μl reaction as described above. One μl of cDNA was the used as template for PCR to amplify CHRFAM7A open reading frame (ORF). The PCR products were purified and cloned into pcDNA3.1 and the identity of the insert was confirmed by DNA sequencing (Retrogen). The primers used were: Sense (5′-AGTCCTCGAGATGCAAAAATATTGCATCT-3′) (SEQ ID NO:9) carrying an XhoI restriction site, Anti-sense (5′-ATTCGGATCCTTACGCAAAGTCTTTGGACACGGC-3′) (SEQ ID NO:10) carrying a BamHI restriction site.

Analyses of the CHRFAM7A Promoter:

The putative CHRFAM7A promoter region spanning from −2363 to +22 relative to the open reading frame ATG start codon was amplified by PCR of genomic DNA isolated from HEK293 cells. The longest fragment was cloned into pGL4 promoter-less luciferase reporter plasmid (Promega) according to the manufacturers specifications and the resulting plasmid, pGL4-CHRFAM7A (2400) was confirmed by DNA sequencing and thereafter referred to F2400 to reflect the size of the fragment.

The primers were: Sense (5′-ATCAGCTAGCTCTAGATAGACAGCATTTTA-3′) (SEQ ID NO:19) containing a NheI restriction site, Anti-sense (5′-GCATAGATCTGGTAGATGCAATATTTTTGCAT-3′) (SEQ ID NO:20) containing a BgIII restriction site.

Three serial 5′ deletion promoter constructs of 1800, 1000, and 500 bp were derived by PCR of the F2400 template using the same anti-sense primer described above, with one of three sense primers to obtain: F1800 (5′-ATCAGCTAGCAAGCCTTCATCAGTGGAAAT-3′) (SEQ ID NO:21), F1000 (5′-ATCAGCTAGCGTATGACTCAAGTCCTTGAC-3′) (SEQ ID NO:22), and F500 (5′-ATCAGCTAGC CTTGCTGTATTCTCTAAACTA-3′) (SEQ ID NO:23).

The fragments generated were cloned into the pGL4 vector to create plasmids f1800, f1000, and f500, which were each sequenced to confirm their identity. These plasmids were then transiently transfected into FHs cells as described below, and luciferase activity was analyzed 30 hours after transfection following the manufacturer's instructions (Promega). Luciferase activity was normalized to protein concentration and the data were presented as relative luciferase activity compared to basal activity in promoter-less pGL4 reporter transfected cells.

Transfections of FHs cells for luciferase activity: The normal human small intestine FHs cells were cultured in 10% DMEM/F12 supplemented with 1× Glutamax, 1× Penicillin/Streptomyxin, and 30 ng/ml EGF and seeded at 1×10⁵ per well in a 12-well plate the day before transfection. The next day, media was refreshed with complete media except Penicillin/Streptomycin two hours before transfection. Transient transfection was performed using Lipofectamine 2000 (Invitrogen). Briefly, 2.5 μl of the Lipofectamine 2000 was added into 50 μl OPTI-MEM (Invitrogen), vortexed for 5 seconds, continued to incubate at room temperature for 5 minutes. One μg plasmid diluted into 50 μl OPTI-MEM was added into the above mixture, vortexed for 5 seconds, and continued to incubate at room temperature for 20 minutes. The DNA-complex was then added drop-wise to cells and cells were continued to incubate for 30 hours. In case of LPS stimulation, LPS was added at 100 ng/ml 3 hours before the 30-hour incubation. Cells were washed with PBS and lysed with 100 μl Passive Lysis buffer at room temperature for 30 minutes with shaking. The lysate was spun down and 10 μl of the supernatant was used for luciferase assay on POLARstar Omega plate reader (BMG LABTECH).

Protein Expression and Immunoblotting:

PC3 cells were seeded at 2×10⁶ per well in a 6-well plate the day before transfection. Cells were transfected with either plasmid encoding human CHRFAM7A variant 1 or control plasmid without insert for 30 hours. Cells were lysed with 300 μl SDS buffer. The lysates were sonicated for 10 bursts at the lowest setting, spun down, and the supernatants were quantitated. Ten μg lysate from each sample was resolved on a 4-12% Bis-Tris gel (Invitrogen) and transferred to PVDF membrane. The membrane was incubated sequentially with Anti-DDK monoclonal antibody at 1:5000 and goat anti-mouse IgG-HRP at 1:10,000 (BioRad) at room temperature for 1 hour respectively. The immunoreactive bands were developed using SuperSignal West Pico Substrate (Thermo Fisher Scientific) and image was acquired using VivoVision IVIS Lumina (Xenogen). Results

Detection and Identification of CHRFAM7A Expression in Human Epithelial Cancer Cells:

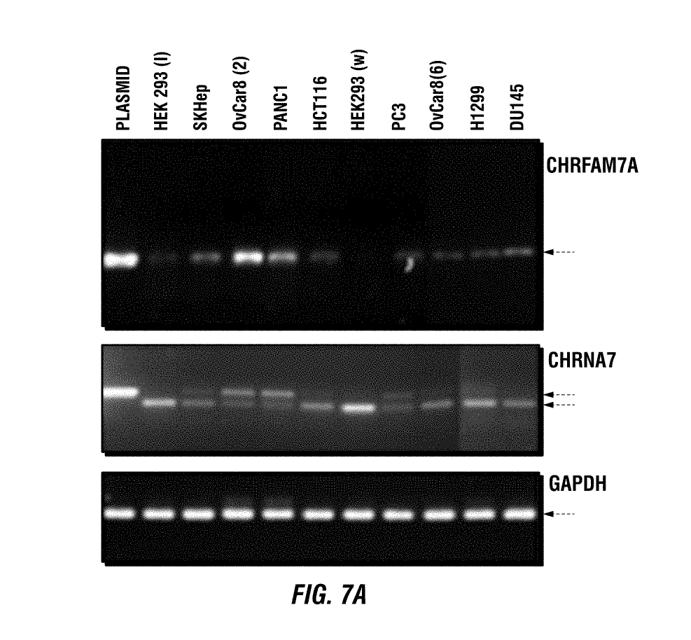

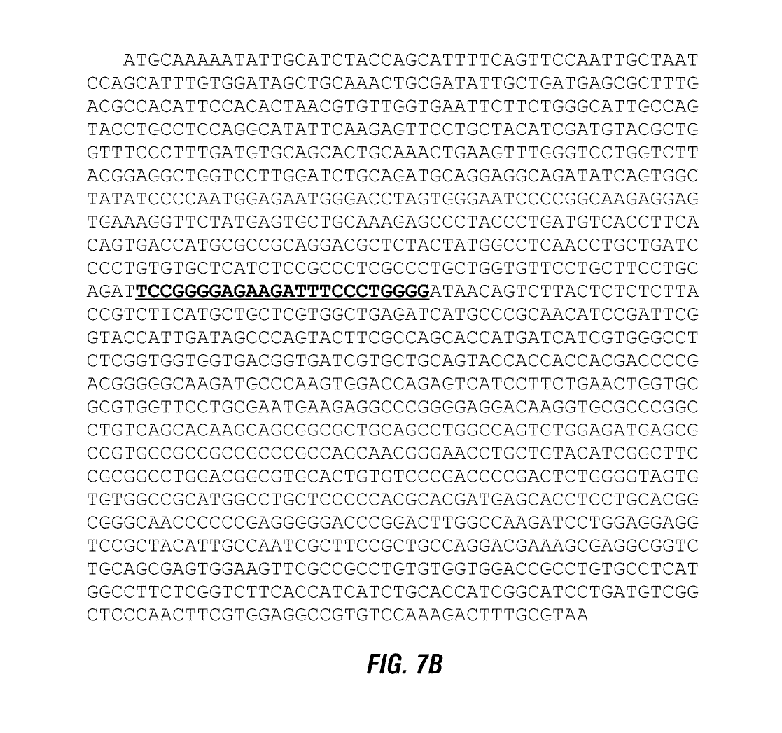

In the course of analyzing the distribution of CHRFAM7A gene expression in human cells, significant CHRFAM7A gene expression in several human epithelial cancer cells lines was detected (FIG. 7). As shown in FIG. 7A, embryonic human kidney cells (HEK293) from multiple sources (I=Invitrogen, W=Wistar), liver cells (SKHep), ovarian cells (OvCar 8) from different sources (1=ATCC, 2=Ciblex Corp), pancreatic cells (PANC1), colon tumor cells (HCT116), lung cells (H1299) and prostate epithelial (PC3, DU145) cancer cell lines all express CHRFAM7A, albeit to different levels. The expression of CHRNA7, the gene encoding the α7nAChR that is common to other species, was assesed. PCR primers were specifically selected to permit the detection of both transcripts 1 and 2. The first (Variant 1) encodes a 118 amino acid sequence in lieu of the 27 amino acid FAM7 sequence found in CHRFAM7A while the second (transcript 2) encodes an additional 22 amino acid insert to produce a 146 amino acid sequence in lieu of the FAM7 sequence found in CHRFAM7A (see FIG. 7D). The differential primer size (66 bp) enables a differentiation after RT-PCR that is not measured by the quantitative RT PCR used. Like CHRFAM7A, the expression of CHRNA7 is variable and some cells appear to exclusively express transcript 2 of CHRNA7 (e.g. HEK293) while others show a preponderance of transcript 2 over transcript 1 (e.g. HCT116, SKHep) and still others (e.g. PANC1) express more CHRNA7 transcript 1. These transcripts encode α7nAChRs that are generated by alternative splicing of CHRNA7 mRNA to produce proteins with distinct amino termini but the physiological significance of this difference is not known. Interestingly, OvCar 8 cells obtained from two different sources (lanes 4 and 9) show a different pattern of transcript 1 and 2 expression between themselves.

One epithelial cell line that showed particularly high CHRFAM7A gene expression was CaCo2 cells (see below). Because CaCo2 cells are commonly used as an in vitro model to study human intestinal epithelial cell growth, barrier permeability and function, using PCR of these and of the normal human small intestine FHs-Int-74 cells was used to amplify, clone and then sequence the epithelial CHRFAM7A transcript (FIG. 7B). When translated, the gut epithelial CHRFAM7A mRNA encodes an open reading frame (ORF) that corresponds to the CHRFAM7A transcript 1 that is found in genomic databases. Translation predicts the existence of a human-specific 411 amino acid CHRFAM7A protein that has a unique terminal 27 amino acids (FIG. 7C) that originates from rearrangement of the ULK sequence of human chromosome 3 when humanoids diverged from primates. This sequence substitutes for the 146 amino acid sequence that was not duplicated from the original CHRNA7 gene on chromosome 15 (FIG. 7D). As expected the remaining sequence (FIG. 7E) is 100% identical to human α7nAChR and derives from the partially duplicated exon 5-10 sequences of CHRNA7(29). This C-terminal peptide sequence contains the monomer channel and transmembrane domains of CHRNA7 so that like CHRNA7, the epithelial CHRFAM7A ORF encodes a protein (48 kDa) that differs from the CHRNA7 (58 kDa) by a distinct amino terminus and molecular weight. The CHRFAM7A transcript 2 present in gene expression databases like ACEVIEW (Thierry-Mieg et al. (2006) “AceView: a comprehensive cDNA-supported gene and transcripts annotation” Gen. biol. 7 Suppl 1, S12 11-14) and purported to encode yet another human-specific variant of CHRNA7 were not detected.

Distribution of CHRFAM7A Expression Transcript in Human Epithelial Cells:

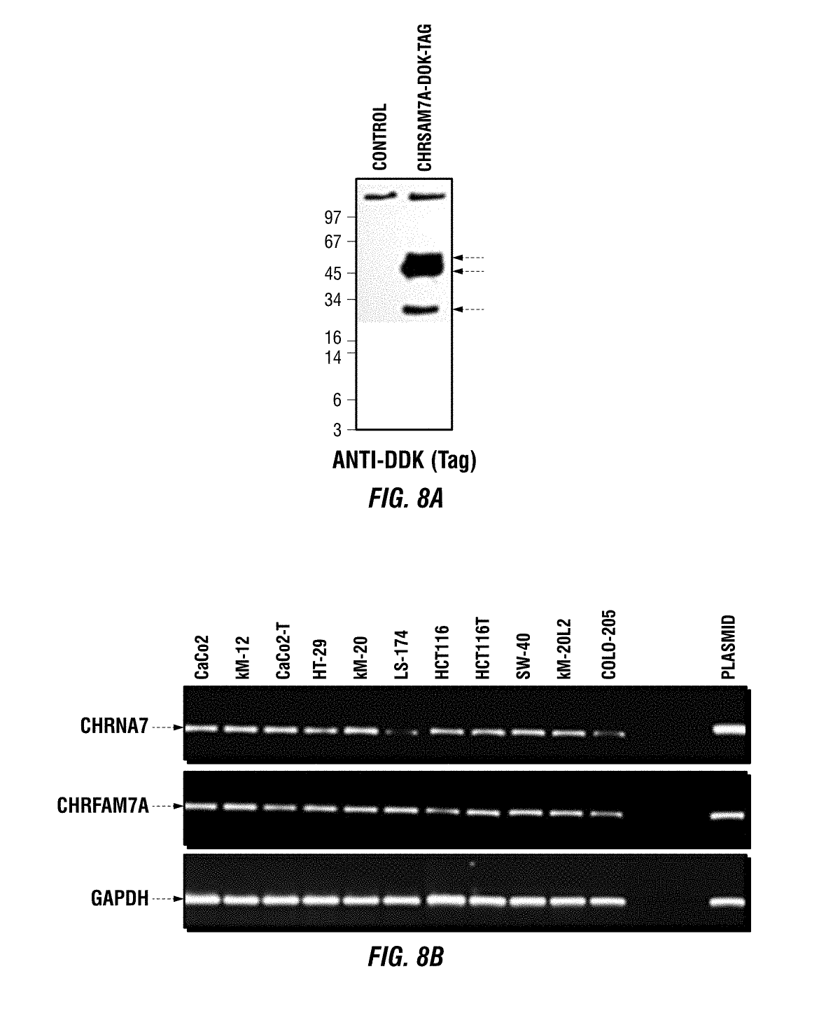

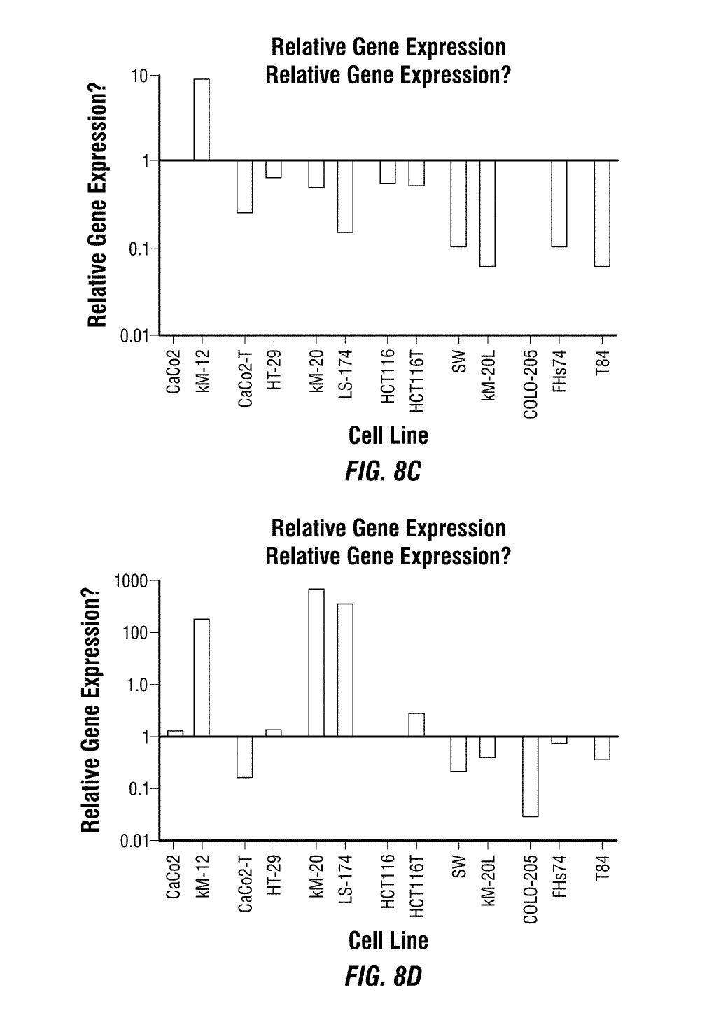

To establish that the epithelial CHRFAM7A ORF can express a CHRFAM7A protein, PC3 prostate cancer epithelial cells were transiently transfected with a plasmid encoding a DDK-tagged CHRFAM7A protein (FIG. 8). Antibodies to DDK were used to detect CHRFAM7A protein in cell lysates and as shown in FIG. 8A, a protein of 48 kDa was readily detected by immunoblotting. Two smaller proteins of 30 and 45 KDa were also observed in cell lysates and are presumably generated by degradation and/or post-translational processing. Their significance, if any is not known.

The extent of CHRFAM7A expression in human colon epithelial cells (FIG. 8B) was evaluated. RT-PCR of RNA prepared from nine different human gut epithelial cell lines show that both CHRFAM7A and CHRNA7 are widely expressed. When assessed by qPCR and normalized to the expression in CaCo2 cells (FIG. 8C), the gut epithelial cell lines had lower levels of CHRNA7 gene expression than CaCo2 cells with the exception of KM12 cells. The differences however were smaller than the differences observed in CHRFAM7A gene expression which varied by from over 100 times higher than the levels found in CaCo2 cells (e.g KM12, KM20 and LS174 cells) to 50 times lower (Colo205 cells).

Regulation of CHRFAM7A Expression in Intestinal Epithelial Cells by Lipopolysaccharide (LPS):

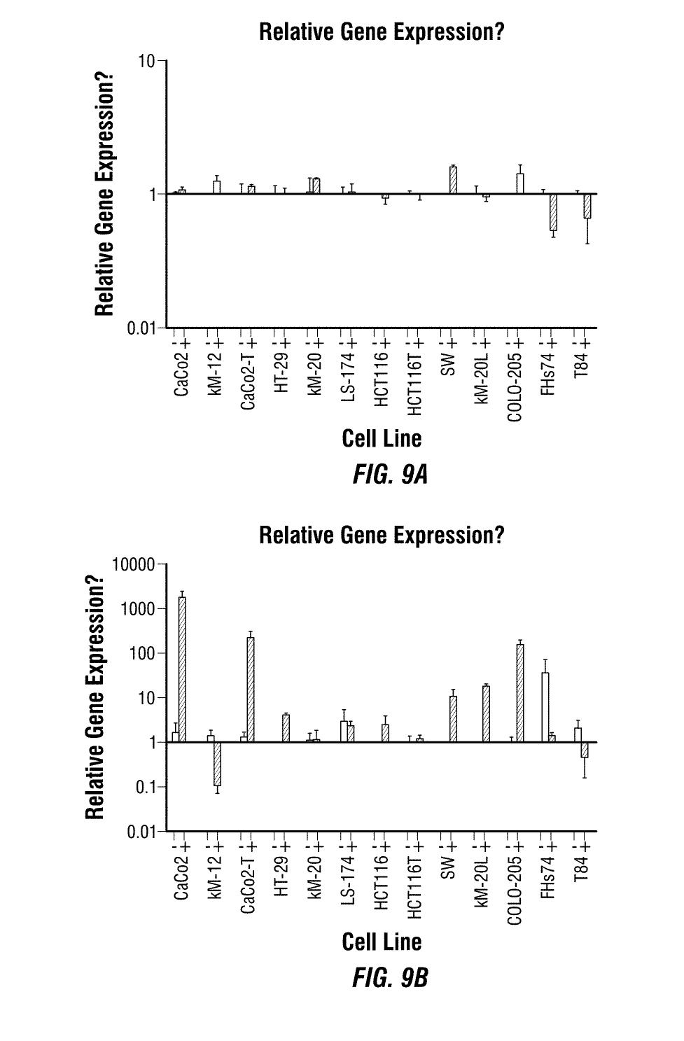

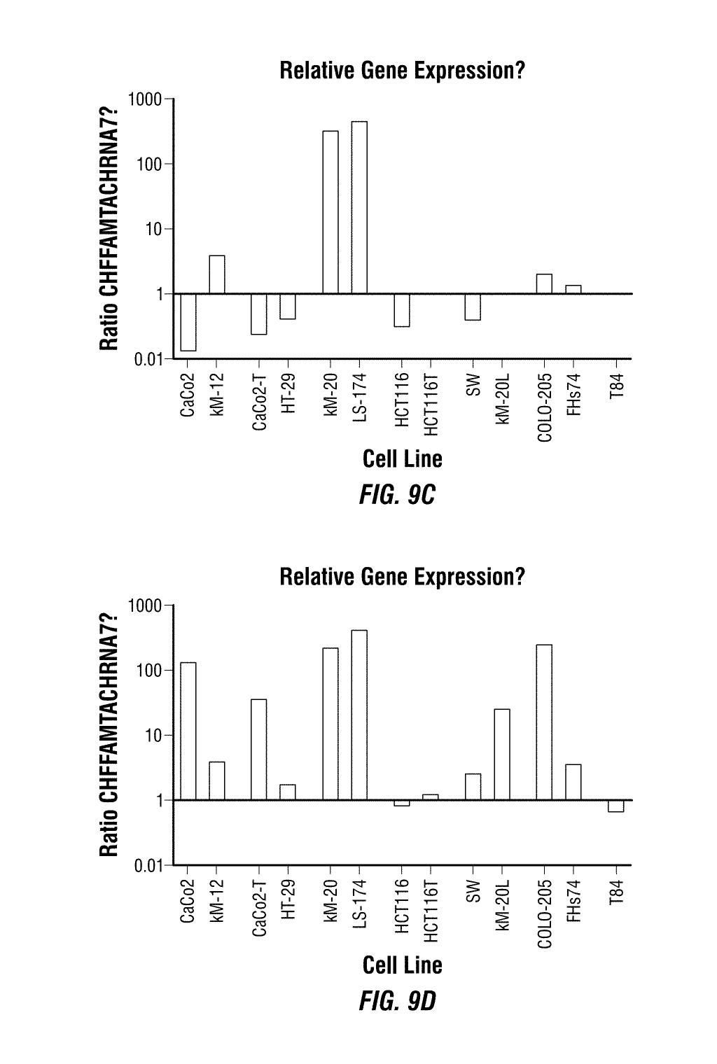

Very little is known regarding the regulation of CHRFAM7A gene expression but at least two groups have reported that LPS inhibits both CHRNA7 and CHRFAM7A in undifferentiated human THP1 cells and human macrophages. In contrast, surprisingly little is published on the effects of LPS treatment in gut epithelial cells presumably because these cells are constitutively exposed to LPS in vivo. As shown in FIG. 9A, the treatment of 11 different epithelial cells with 100 ng of LPS for 3 hours has little effect on the expression of CHRNA7. In several instances (CaCo2, KM12, KM20, SW, Colo205), a small increase in CHRNA7 gene expression was detected, but it was generally less than two-fold. In two instances (FHs and T84 cells), a small (30%) decrease in gene expression was observed.

In contrast, CHRFAM7A appeared highly responsive to 100 ng/ml LPS (FIG. 9B). Two cell lines (KM12 and T84 cells) showed decreased CHRFAM7A in response to LPS treatment but the treatment of all other cell lines resulted in increases of CHRFAM7A gene expression from 200 to 1200 fold (CaCo2 cells) or 5 to 100 fold (HT29, SW, KM20L, Colo205 cells). When normalized to the expression of CHRNA7, the basal gene expression of CHRNA7 and CHRFAM7A was highly variable with five cell lines expressing more CHRFAM7A then CHRNA7 (KM12, KM20, LS174, CoLo205 and FHs cells), another five lines showing the opposite pattern (CaCo2, CaCo2T, HT29, HCT116 and SW cells) and another 3 lines (HCT116T, KM20L and T84 cells) being about equal (FIG. 9C). The overall effect of treating these gut epithelial cells with LPS however was to increase the relative expression of CHRFAM7A from 2-200 fold over that of CHRNA7 expression depending on the cell line (FIG. 9C) although three cell lines (HCT116T, KM20L and T84) appeared unchanged. Although the biological significance of the differential effect of LPS on CHRFAM7A and CHRNA7 is not known, the net effect of LPS treatment is to change the profile of epithelial cells so that they have increased CHRFAM7A compared to the levels of CHRNA7 (FIG. 9D).

Characterization of CHRFAM7A Promoter.

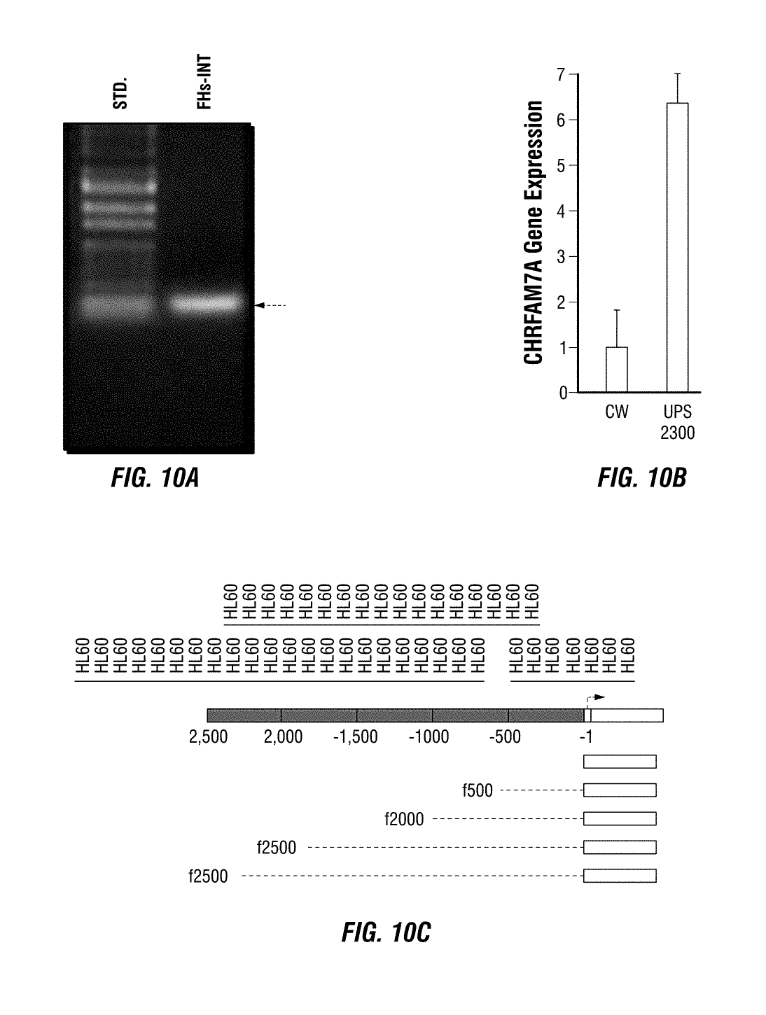

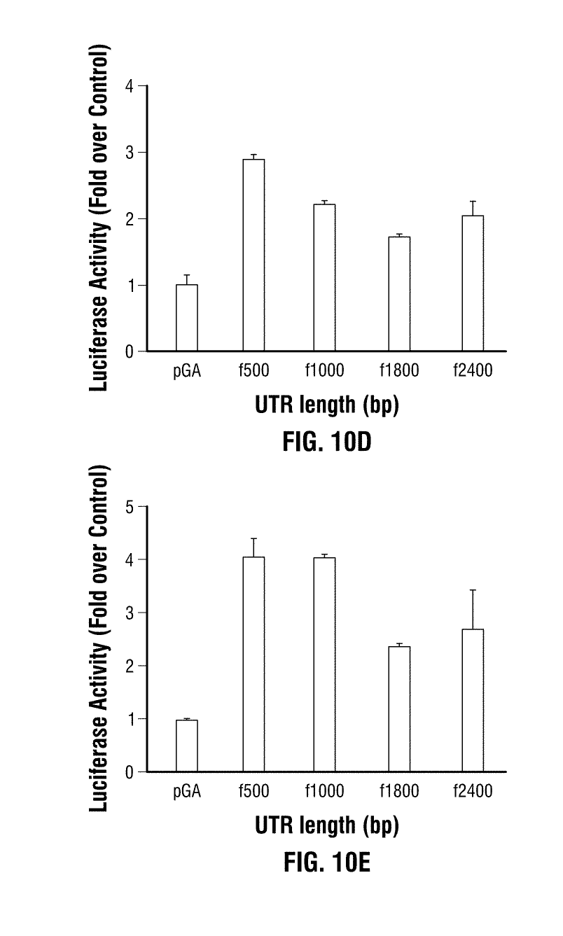

Because FHs cells are derived from normal, untransformed human fetal small intestine epithelial cells, they express CHRFAM7A (FIG. 10A) and respond to LPS (FIG. 10B), they were selected to analyze the CHRFAM7A UTR for promoter activity. Bioinformatic analyses of the expected CHRFAM7A promoter region (FIG. 10C) revealed the presence of numerous consensus binding sites for transcription factors (e.g. NFκb) that have been implicated in LPS responsiveness (Sweet et al. (1996) “Endotoxin signal transduction in macrophages” J. of leukocyte biol. 60, 8-26; Hawiger (2001) “Innate immunity and inflammation: a transcriptional paradigm” Immuno. Res. 23, 99-109; Pasparakis (2008) “IKK/NF-kappaB signaling in intestinal epithelial cells controls immune homeostasis in the gut” Mucosal immuno. 1 Suppl 1, S54-57). Four fragments ranging from 500 bp to 2,400 bp were prepared from the CHRFAM7A gene as described in materials and methods. The fragments were cloned into the promoter-less pGL4 vector to create plasmids 2400, f1800, f1000, and f500 and transiently transfected into FHs cells. Luciferase activity was analyzed 30 hours later, normalized to protein concentration and analyzed relative to basal luciferase activity generated by promoter-less pGL4 reporter transfected cells (FIG. 10C). Each fragment was analyzed for its ability to generate luciferase activity after transfection into control (FIG. 10D) or LPS-stimulated (FIG. 10E) FHs cells. As shown, luciferase activity was increased over baseline in the fragment containing the first 500 bp sequence 5′ of the CHRFAM7A open reading frame. Extensions of this sequence do not increase luciferase and instead, fragments extending beyond 500 bp to 2.4 kb are inhibitory and show decreased luciferase activity. A very similar profile is observed with LPS treatment (FIG. 10D) and increased gene expression might be attributed to disinhibition of elements binding −500 bp to −1000 bp, which no longer shows inhibition of gene expression. Together, these data indicate a regulatory function for the non-coding exons (E, D and C) that translocated with Exons B and A (FIG. 7C) to form the CHRFAM7A open reading frame.

### Discussion

While there are many homologs and orthologs of human genes that are found in other species, some are taxonomically-restricted gene (TRG) paralogs that are shared within taxa (e.g. primates) while others, are species-specific, for example unique to humans. One selectively human-specific gene, called CHRFAM7A, was studied and demonstrated to be widely expressed in human epithelial cells (FIGS. 7 and 8). It was also shown that its sequence is unique from the CHRNA7 “parent” gene from which it is partially duplicated and that it is found in other species, including humans. It was shown that the human-specific CHRFAM7A gene expressed in epithelial cells has differential responsiveness from CHRNA7 to a trophic stimulus (FIG. 9) and that the promoter regulation of the newly evolved CHRFAM7A from its ancestral “older” parent CHRNA7 gene is distinct. Finally, differential regulation of CHRFAM7A can be tracked to a promoter sequence 500 bp to 1000 bp from the CHRFAM7A open reading frame (FIG. 10). In light of recent reports demonstrating that CHRFAM7A can modulate the expression, biological function and activity of CHRNA7, the findings presented imply the existence of a human-specific process in human epithelial cells that controls the epithelial cell response to LPS. While a similar process might exist in other species, it is not mediated by CHRFAM7A and the exact nature and physiological significance of this human-specific mechanism requires further investigation.

There are more than 300 human-specific genes that have been identified to date and they are believed to arise from either segmental duplication of pre-existing genes, species-specific alternative splicing, endogenization of retroviruses or mutational events that occurred during humanoid divergence from primates. While the presence of human-specific genes might explain the differential responsiveness of human cells to trophic stimuli that is sometimes observed in animal models, their role in human physiology has been under-investigated. First and foremost, it has been necessary to determine whether human-specific genes are expressed or pseudo-genes, to determine where they are expressed, study the regulation of their expression and then, establish the potential physiological and/or pathophysiological consequence of their expression. To date, it is only known that “new” human genes are over-represented in complex disease thereby implying they might participate in diseases “characteristically human”.

A case in point is the human chromosome 15q13-14 locus, which encodes the human CHRNA7 gene and that results in the expression of the α7nAChR, which has long been associated with human mental illness. This locus has undergone significant rearrangement since human divergence from primates 9-12 million years ago and one genetic rearrangement includes the emergence of a new, distinct, partially duplicated, and rearranged gene called CHRFAM7A. While the CHRFAM7A gene is structurally related to CHRNA7 and shares six duplicated exons 5-10 of CHRNA7, it has also acquired exons A-E of the FAM7 pseudo-gene that itself, arose from the UL kinase gene on human chromosome 3. Since its discovery, numerous studies have shown that CHRFAM7A is expressed in neuronal cells and several genomic analyses have examined CHRFAM7A gene expression in neuropsychiatric disorders including schizophrenia, bipolar disorder, and autism. While its mechanism of action remains unclear in the CNS, its product is presumed to behave like CHRNA7 and as a ligand-gated channel. However, the existence of a mutant form of CHRFAM7A called Δ2 bp-CHRFAM7A has been implicated in severe mental illness implying that CHRFAM7A may play a heretofore unknown, but significant, function in human cognitive function.

CHRFAM7A expression was demonstrated in inflammatory cells soon after its identification in the CHRNA7 locus of human chromosome 15, but its significance and function is not understood. In leukocyte cell lines like THP1 cells and in normal human monocytes, CHRFAM7A gene expression is reported to parallel that of CHRNA7. Unfortunately, primer cross hybridization and antibody cross reactivity have confounded several studies that purport to measure CHRFAM7A gene expression or CHRFAM7A protein in cell lysates. Other more recent studies point to CHRFAM7A playing roles as a dominant negative inhibitor of the α7nAChR, altering ligand (nicotine) signaling or interfering with CHRNA7 gene expression, assembly and cell signaling functions. Its presence and regulation, let alone activity, in epithelial cells has not been reported and is unknown.

Although the natural ligand(s) for CHRFAM7A is not known, there is compelling evidence that CHRFAM7A could play a role in epithelial cell biology, specifically in regulating α7nAChR activity. First, CHRFAM7A has the capacity to form cell surface hetero-polymers with wild type α7nAChR. Second, it is reported to exert a dominant negative effect on α7nAChR and regulate the appearance of α7nAChR on the cell surface. In as much as a role for α7nAChRs in epithelial cell homeostasis now appears unequivocal (Maouche et al. (2013) “Contribution of alpha7 nicotinic receptor to airway epithelium dysfunction under nicotine exposure” Proc. of the Nat. Ac. of Sci. of the U. S. of Am. 110, 4099-4104), CHRFAM7A might therefore confer a “human-selective” responsiveness to gut epithelium. Interestingly, gut epithelial cells appear refractory to the inflammatory effects of LPS that are observed in human leukocytes and are prototypic regulators of barrier function and immune homeostasis. The intestinal epithelium however is normally constitutively exposed to intraluminal bacterial products, including LPS, so that a selective up-regulation of CHRFAM7A in intestinal epithelial cells could conceivably contribute to a species-specific resistance to inflammation. This hypothesis might help explain elevated CHRFAM7A gene expression in normal human gut epithelium reported here. Interestingly, the CHRFAM7A gene is absent or only present as a single copy in 5-15% of humans. It is therefore likely that CHRFAM7A may provide a protective effect to epithelial cells. With CHRFAM7A expression in both gut epithelium and leukocytes, it will be interesting to mine public and private gene expression databases for possible changes in CHRFAM7A expression that might link its expression to the onset, development and resolution of clinical conditions including inflammatory bowel diseases and cancer. Its presence could also have potential implications for drug development and drug responsiveness.

Finally, it is noteworthy that a differential regulation of CHRFAM7A could provide humans with a species-specific response to inflammatory stimuli that is not replicated in animal models of human disease. To this end, studies of the CHRFAM7A locus on human chromosome 15 have historically focused on its association with schizophrenia, a prototypically human-specific disease, and not inflammation or epithelial biology. Yet interestingly, schizophrenia is associated with changed risk for colon cancer and irritable bowel syndromes and a 2 bp mutation in CHRFAM7A. These disease targets are both affected by nicotinic AChR activation and link nicotine to epithelial cell proliferation and gut epithelial permeability. Together, these associations underscore the premise that the identification of a human-specific CHRFAM7A in human gut epithelial is only the first step towards defining its potential physiological and pathophysiological function(s), and understanding the molecular basis to the emergence, selection and retention of human genes in the human genome during human speciation.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

What is claimed is:

1. A method for treating an inflammatory response in leukocytes for a clinical disease comprising administering to a subject in need an effective amount of a pharmaceutical composition comprising an agent that increases expression or activity of CHRFAM7A in said leukocytes.

2. The method of claim 1, wherein said agent is a lipopolysaccharide or a functional fragment thereof.

3. The method of claim 1, wherein said agent alters α7nAchR binding.

4. The method of claim 1, wherein said agent alters leukocyte adhesion.

5. The method of claim 1, wherein said agent alters expression or activity of human-specific genes (HSGs) or taxonomically-restricted genes (TRGs) associated with focal adhesion, leukocyte trans-epithelial migration, or cancer.

6. The method of claim 1, wherein said clinical disease is selected from the group consisting of sepsis, systemic inflammatory response to injury, and pancreatitis.

7. A method for treating inflammation in epithelial cells for a clinical disease comprising administering to a subject in need an effective amount of a pharmaceutical composition comprising an agent that increases expression or activity of CHRFAM7A in said epithelial cells.

8. The method of claim 7, wherein said agent is a lipopolysaccharide or a functional fragment thereof.

9. The method of claim 7, wherein said agent alters α7nAchR binding.

10. The method of claim 7, wherein said epithelium comprises gut epithelial cells.

11. The method of claim 10, wherein said gut epithelial cells comprise intestinal or colon epithelial cells.

12. The method of claim 7, wherein said clinical disease is selected from the group consisting of sepsis, trauma injury, burn injury, inflammatory bowel disease, necrotizing enterocolitis, enteritis, and infectious colitis.

13. A pharmaceutical composition comprising:
a therapeutic agent in an amount effective to increase expression or activity of CHRFAM7A in leukocytes or epithelial cells; and
at least one pharmaceutically acceptable excipient.

14. The composition of claim 13, wherein said therapeutic agent is a lipopolysaccharide or a functional fragment thereof.

15. The composition of claim 13, wherein said therapeutic agent is a ligand for CHRFAM7A promoter region.

16. The composition of claim 15, wherein said ligand is an antibody.

17. The composition of claim 15 wherein said ligand is a polypeptide.

18. The composition of claim 15, wherein said ligand is an oligonucleotide.

19. The composition of claim 13, wherein said at least one pharmaceutically acceptable excipient includes a pharmaceutically acceptable carrier.

20. The composition of claim 13, which further comprises at least one additional active ingredient.


SEQUENCE LISTING (23 sequences)

SEQ ID NO: 1 - DNA, 560 bp, Homo sapiens
catagctcccgccaagtcctcggtgccccttgccattttccagccgcgctcccacgagggtcacggcggcggggagaggtggagccgcgagagctcggccgggggccccgcctggtggtcgcggccatgacagcggctcgggacaggctccttttccgcgcccctcccgccggaggtgaggggaagatgtccatgtcagggttcaaggccaaaccgaagttactggcctctattttccaggagaaccaggagccacagccgcggctcacgccccaccgcaacattaagattacaagtggacacctgagtcagcaggacccggaatcccagatgagagagcttatctacacgactcagatcttgttgtcacccccattattgacaatccaaaggtgcagaaagcactctgacaaataatgaaacaaccaccatcggttaaatttgatgcaaaaatattgcatctaccagcattttcagttccaattgctaatccagcatttgtggatagctgcaaactgtgatattgctgatgagcgctttgacgccacattccacactaa

SEQ ID NO: 2 - protein, 27 aa, Homo sapiens
MQKYCIYQHFQFQLLIQHLWIAANCDI

SEQ ID NO: 3 - protein, 146 aa, Homo sapiens
MRCSPGGVWLALAASLLHGKATASPPSTPPWDPGHIPGASVRPAPGPVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNS

SEQ ID NO: 4 - protein, 384 aa, Homo sapiens
ADERFDATFHTNVLVNSSGHCQYLPPGIFKSSCYIDVRWFPFDVQHCKLKFGSWSYGGWSLDLQMQEADISGYIPNGEWDLVGIPGKRSERFYECCKEPYPDVTFTVTMRRRTLYYGLNLLIPCVLISALALLVFLLPADSGEKISLGITVLLSLTVFMLLVAEIMPATSDSVPLIAQYFASTMIIVGLSVVVTVIVLQYHHHDPDGGKMPKWTRVILNWCAWFLRMKRPGEDKVRPACQHKQRRCSLASVEMSAVAPPPASNGNLLYIGFRGLDGVHCVPTPDSGVVCGRMACSPTHDEHLLHGGQPPEGDPDLAKILEEVRYIANRFRCQDESEAVCSEWKFAACVVDRLCLMAFSVFTIICTIGILMSAPNFVEAVSKDFA

SEQ ID NO: 5 - DNA, 1238 bp, Homo sapiens
atgcaaaaatattgcatctaccagcattttcagttccaattgctaatccagcatttgtggatagctgcaaactgcgatattgctgatgagcgctttgacgccacattccacactaacgtgttggtgaattcttctgggcattgccagtacctgcctccaggcatattcaagagttcctgctacatcgatgtacgctggtttccctttgatgtgcagcactgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccctgatgtcaccttcacagtgaccatgcgccgcaggacgctctactatggcctcaacctgctgatcccctgtgtgctcatctccgccctcgccctgctggtgttcctgcttcctgcagattccggggagaagatttccctggggataacagtcttactctctcttaccgtcttcatgctgctcgtggctgagatcatgcccgcaacatccgattcggtaccattgatagcccagtacttcgccagcaccatgatcatcgtgggcctctcggtggtggtgacggtgatcgtgctgcagtaccaccaccacgaccccgacgggggcaagatgcccaagtggaccagagtcatccttctgaactggtgcgcgtggttcctgcgaatgaagaggcccggggaggacaaggtgcgcccggcctgtcagcacaagcagcggcgctgcagcctggccagtgtggagatgagcgccgtggcgccgccgcccgccagcaacgggaacctgctgtacatcggcttccgcggcctggacggcgtgcactgtgtcccgaccccgactctggggtagtgtgtggccgcatggcctgctcccccacgcacgatgagcacctcctgcacggcgggcaaccccccgagggggacccggacttggccaagatcctggaggaggtccgctacattgccaatcgcttccgctgccaggacgaaagcgaggcggtctgcagcgagtggaagttcgccgcctgtgtggtggaccgcctgtgcctcatggccttctcggtcttcaccatcatctgcaccatcggcatcctgatgtcggctcccaacttcgtggaggccgtgtccaaagactttgcgtaa

SEQ ID NO: 6 - protein, 27 aa, Homo sapiens
MQKYCIYQHFQFQLLIQHLWIAANCDI

SEQ ID NO: 7 - protein, 146 aa, Homo sapiens; identical to SEQ ID NO: 3

SEQ ID NO: 8 - protein, 384 aa, Homo sapiens; identical to SEQ ID NO: 4

SEQ ID NO: 9 - DNA, 29 bp, Artificial Sequence
  Description of Artificial Sequence Synthetic primer
agtcctcgagatgcaaaaatattgcatct

SEQ ID NO: 10 - DNA, 34 bp, Artificial Sequence
  Description of Artificial Sequence Synthetic primer
attcggatccttacgcaaagtctttggacacggc

SEQ ID NO: 11 - DNA, 19 bp, Artificial Sequence
  Description of Artificial Sequence Synthetic primer
atagctgcaaactgcgata

SEQ ID NO: 12 - DNA, 21 bp, Artificial Sequence
  Description of Artificial Sequence Synthetic primer
cagcgtacatcgatgtagcag

SEQ ID NO: 13 - DNA, 20 bp, Artificial Sequence
  Description of Artificial Sequence Synthetic primer
acatgcgctgctcgccggga

SEQ ID NO: 14 - DNA, 21 bp, Artificial Sequence
  Description of Artificial Sequence Synthetic primer
gattgtagttcttgaccagct

SEQ ID NO: 15 - DNA, 23 bp, Artificial Sequence
  Description of Artificial Sequence Synthetic primer
catgagaagtatgacaacagcct

SEQ ID NO: 16 - DNA, 22 bp, Artificial Sequence
  Description of Artificial Sequence Synthetic primer
agtccttccacgataccaaagt

SEQ ID NO: 17 - DNA, 24 bp, Artificial Sequence
  Description of Artificial Sequence Synthetic oligonucleotide
gcaggtactggcaatgcccagaag

SEQ ID NO: 18 - DNA, 25 bp, Artificial Sequence
  Description of Artificial Sequence Synthetic oligonucleotide
tagtgtggaatgtggcgtcaaagcg

SEQ ID NO: 19 - DNA, 30 bp, Artificial Sequence
  Description of Artificial Sequence Synthetic primer
atcagctagctctagatagacagcatttta

SEQ ID NO: 20 - DNA, 32 bp, Artificial Sequence
  Description of Artificial Sequence Synthetic primer
gcatagatctggtagatgcaatatttttgcat

SEQ ID NO: 21 - DNA, 30 bp, Artificial Sequence
  Description of Artificial Sequence Synthetic primer
atcagctagcaagccttcatcagtggaaat

SEQ ID NO: 22 - DNA, 30 bp, Artificial Sequence
  Description of Artificial Sequence Synthetic primer
atcagctagcgtatgactcaagtccttgac

SEQ ID NO: 23 - DNA, 31 bp, Artificial Sequence
  Description of Artificial Sequence Synthetic primer
atcagctagccttgctgtattctctaaacta